SEQUENCE LISTING (26 sequences)

SEQ ID NO: 1 - DNA, 81 bp, Artificial Sequence
  Synthetic sequence
aggucaaacccuuaagacaucugaagcugcaaccuggccuuugguguugaaauaggaagguuuaaggagaaucuaagcauu

SEQ ID NO: 2 - DNA, 83 bp, Artificial Sequence
  Synthetic sequence
aaugcuuagauucuccuuaaaccuuccuauuucaacaccaaaggccagguugcagcuucagaugucuuaaggguuugaccuga

SEQ ID NO: 3 - DNA, 25 bp, Artificial Sequence
  Synthetic sequence
aagguuuaaggagaaucuaagcauu

SEQ ID NO: 4 - DNA, 27 bp, Artificial Sequence
  Synthetic sequence
aaugcuuagauucuccuuaaaccuucc

SEQ ID NO: 5 - DNA, 25 bp, Artificial Sequence
  Synthetic sequence
aggucaaacccuuaagacaucugaa

SEQ ID NO: 6 - DNA, 27 bp, Artificial Sequence
  Synthetic sequence
uucagaugucuuaaggguuugaccuga

SEQ ID NO: 7 - DNA, 25 bp, Artificial Sequence
  Synthetic sequence
ggucaaacccuuaagacaucugaag

SEQ ID NO: 8 - DNA, 27 bp, Artificial Sequence
  Synthetic sequence
cuucagaugucuuaaggguuugaccug

SEQ ID NO: 9 - DNA, 25 bp, Artificial Sequence
  Synthetic sequence
  misc_feature (1)..(23): ribonucleotide
  misc_feature (24)..(25): deoxyribonucleotide
aagguuuaaggagaaucuaagcatt

SEQ ID NO: 10 - RNA, 27 nt, Artificial Sequence
  Synthetic sequence
aaugcuuagauucuccuuaaaccuucc

SEQ ID NO: 11 - DNA, 25 bp, Artificial Sequence
  Synthetic sequence
  misc_feature (1)..(23): ribonucleotide
  misc_feature (24)..(25): deoxyribonucleotide
aggucaaacccuuaagacaucugaa

SEQ ID NO: 12 - RNA, 27 nt, Artificial Sequence
  Synthetic sequence
uucagaugucuuaaggguuugaccuga

SEQ ID NO: 13 - DNA, 25 bp, Artificial Sequence
  Synthetic sequence
  misc_feature (1)..(23): ribonucleotide
  misc_feature (24)..(25): deoxyribonucleotide
ggucaaacccuuaagacaucugaag

SEQ ID NO: 14 - RNA, 27 nt, Artificial Sequence
  Synthetic sequence
cuucagaugucuuaaggguuugaccug

SEQ ID NO: 15 - DNA, 40 bp, Artificial Sequence
  Synthetic sequence
ugcguucacguuuauucacauuuuugaauugagcaugagc

SEQ ID NO: 16 - DNA, 17 bp, Artificial Sequence
  Synthetic sequence
guacauucuagauacgc

SEQ ID NO: 17 - DNA, 2425 bp, Artificial Sequence
  Synthetic sequence
ggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 18 - DNA, 16 bp, Artificial Sequence
  Synthetic sequence
gctgacatccaaatag

SEQ ID NO: 19 - DNA, 87 bp, Artificial Sequence
  Synthetic sequence
  misc_feature (24)..(63): 40 nucleotide sequences formed by equimolar incorporation of A, G, C, and U at each position
gggagacaagaataaacgctcaannnnnnnnnnnnnnnnnnnnnnnnnnnnnnnnnnnnnnnnttcgacaggaggctcacaacaggc

SEQ ID NO: 20 - DNA, 87 bp, Artificial Sequence
  Synthetic sequence
gggagacaagaauaaacgcucaaugcguucacguuuauucacauuuuugaauugagcaugagcuucgacaggaggcucacaacaggc

SEQ ID NO: 21 - DNA, 81 bp, Artificial Sequence
  Synthetic sequence
  misc_feature (1)..(40): 40-nt-long random sequence
  misc_feature (64)..(65): C3 carbon linker
nnnnnnnnnnnnnnnnnnnnnnnnnnnnnnnnnnnnnnnnuucgacaggaggcucacaacaggcguacauucuagauacgc

SEQ ID NO: 22 - DNA, 19 bp, Artificial Sequence
  Synthetic sequence
gagauugaccagcaguaua

SEQ ID NO: 23 - DNA, 18 bp, Artificial Sequence
  Synthetic sequence
ccaacaacccaagaaugu

SEQ ID NO: 24 - DNA, 19 bp, Artificial Sequence
  Synthetic sequence
caacagauugccugcauug

SEQ ID NO: 25 - DNA, 21 bp, Artificial Sequence
  Synthetic sequence
cagcctctctgcagaattcaa

SEQ ID NO: 26 - DNA, 21 bp, Artificial Sequence
  Synthetic sequence
ggcugguaauuuauauaautt

(19) United States
(12) Patent Application Publication
(10) Pub. No.: US 2024/0043837 A1
(43) Pub. Date: Feb. 8, 2024

(54) MODULATION OF SIGNAL TRANSDUCER AND ACTIVATOR OF TRANSCRIPTION 3 (STAT3) EXPRESSION

(71) Applicant: NATIONAL UNIVERSITY OF SINGAPORE, Singapore (SG)

(72) Inventors: Boon Cher GOH, Singapore (SG); Li Ren KONG, Singapore (SG)

(21) Appl. No.: 18/040,503

(22) PCT Filed: Aug. 6, 2021
(86) PCT No.: PCT/SG2021/050462

§ 371 Date: Feb. 3, 2023

(30) Foreign Application Priority Data
Aug. 6, 2020 (SG) 10202007545P

(51) Int. Cl. C12N 15/113; A61K 31/713; A61P 35/00; A61K 47/54

(52) U.S. Cl. CPC C12N 15/113; A61K 31/713; A61P 35/00; A61K 47/549; A61K 47/555

(57) ABSTRACT

The present invention relates to nucleic acids, double stranded nucleic acids (dsNAs), and agents for inhibiting expression of STATS. The present invention also includes nanoparticles comprising the nucleic acids, the dsNAs, and/or the agents as well as methods of treating cancer using the nucleic acids, the double stranded nucleic acids (dsNAs), the agents and/or the nanoparticles as disclosed herein. In one embodiment, the target region is within the 3′-UTR region of STATS mRNA.

### CROSS-REFERENCE TO RELATED APPLICATIONS

This application claims the benefit of priority of Singapore provisional application No. 10202007545P, filed 6 Aug. 2020, the contents of it being hereby incorporated by reference in its entirety for all purposes.

### FIELD OF THE INVENTION

The present invention generally relates to the fields of molecular biology, cell biology and biotechnology. More particularly, the present invention relates to nucleic acids for inhibiting expression of signal transducer and activator of transcription 3 (STAT3), and uses of the nucleic acids.

### BACKGROUND OF THE INVENTION

The STAT (signal transducers and activators of transcription) family of proteins are DNA-binding proteins that play a dual role in signal transduction and activation of transcription. The activities of the STATs are modulated by various cytokines and mitogenic stimuli. Binding of a cytokine to its receptor results in the activation of Janus protein tyrosine kinases (JAKs) associated with these receptors. This in turn, phosphorylates STAT, resulting in translocation to the nucleus and transcriptional activation of STAT responsive genes. Phosphorylation on a specific tyrosine residue on the STATs results in their activation, resulting in the formation of homodimers and/or heterodimers of STAT which bind to specific gene promoter sequences. Events mediated by cytokines through STAT activation include cell proliferation and differentiation and prevention of apoptosis.

STAT3 is expressed in most cell types. Constitutive activation of STAT3 signaling is a feature of many cancers, and STAT3 promotes cancer inflammation. STAT3 is also a key node in many growth factor receptor mediated oncogenic signaling pathways. Thus, STAT3 is an attractive target for therapeutic intervention in cancers with activated STAT3 signaling. Currently, FDA-approved agents targeting the STAT3 activation cascade include monoclonal antibody against the interleukin-6 receptor (tocilizumab, siltuximab), and JAK tyrosine kinase inhibitors (tofacitinib, ruxolitinib), but these are not direct STAT3 inhibitors. Development of STAT3 inhibitors has been hampered by the lack of a tyrosine kinase domain on STAT3, as well as the close homology of STAT3 to other members of the STAT signaling pathway. Thus, there remains an unmet need for therapeutic compositions and methods targeting expression of STAT3, and disease processes associated therewith.

### SUMMARY OF THE INVENTION

In one aspect, there is provided a nucleic acid comprising an oligonucleotide strand of 15-80 nucleotides in length, wherein said oligonucleotide strand is at least 80% complementary to a target STAT3 mRNA sequence along at least 15 nucleotides of said oligonucleotide strand, wherein the target STAT3 mRNA sequence has the sequence of

(SEQ ID NO: 1)
AGGUCAAACCCUUAAGACAUCUGAAGCUGCAACCUGGCCUUUGGUGUUG
AAAUAGGAAGGUUUAAGGAGAAUCUAAGCAUU.

In another aspect, there is provided a double stranded nucleic acid (dsNA) for inhibiting expression of STAT3, comprising a sense strand and an antisense strand forming a double stranded region, wherein the antisense strand is at least 80% complementary to a target STAT3 mRNA sequence along at least 15 nucleotides of said antisense strand, wherein the target STAT3 mRNA sequence has the sequence of SEQ ID NO: 1; and wherein the sense strand is at least 80% complementary to the sequence AAUGCUUAGAUUCUCCUUAAACCUUCCUAUUUCAACACCAAAGGCCAGGUUGC AGCUUCAGAUGUCUUAAGGGUUUGACCUGA (SEQ ID NO: 2), along at least 15 nucleotides of said sense strand.

In one aspect, there is provided an agent for inhibiting expression of STAT3, comprising an aptamer and a double strand nucleic acid (dsNA), said dsNA comprising a sense strand and an antisense strand; wherein the aptamer is attached to one end of the antisense strand via a linker; wherein the aptamer comprises a sequence which differs by no more than 3 nucleotides from SEQ ID NO: 15; and wherein the antisense strand comprises at least 15 contiguous nucleotides differing by no more than 3 nucleotides from a nucleotide sequence selected from the group consisting of SEQ ID NO: 10 SEQ ID NOs: 12, and SEQ ID NOs: 14.

In one aspect, there is provided a nanoparticle comprising the nucleic, the dsNA, and/or the agent as disclosed herein.

In one aspect, there is provided a method of treating cancer in a subject, comprising administering an effective amount of the nucleic acid, the dsNA, the agent, and/or the nanoparticle as disclosed herein.

In another aspect, there is provided the nucleic acid, the dsNA, the agent, and/or the nanoparticle as disclosed herein for use in therapy.

In another aspect, there is provided use of the nucleic acid, the dsNA, the agent, and/or the nanoparticle as disclosed herein in the manufacture of a medicament for treating cancer.

### BRIEF DESCRIPTION OF THE DRAWINGS

The invention will be better understood with reference to the detailed description when considered in conjunction with the non-limiting examples and the accompanying drawings, in which:

FIG. 1 is a diagram depicting the region targeted by commercially available STAT3 siRNAs. Five functionally validated sequences (Dh1, Dh3, Dh4, Q7 and Q8) were purchased from two companies (Dh and Q). As shown in the diagram, all five commercially available siRNAs target the coding region of STAT3.

FIG. 2 shows the results of RT-qPCR quantification of the mRNA level of STAT1, STAT2, STAT3, STAT4, STAT5A and STAT5B in H2170 cancer cell lines transfected with the different commercially available STAT3 siRNAs (Dh1, Dh3, Dh4, Q7 and Q8). 50nM of each siRNA was used, and JetPrime Transfection reagent was used according to manufacturer's protocol. Total RNA was harvested 48 hours post-transfection. GAPDH was used as endogenous control. The results show that each of the five commercially available STAT3 siRNA sequences exhibit non-specific inhibition of other STAT isoforms in H2170 cell line.

FIG. 3 is a diagram depicting the region targeted by the STAT3 DsiRNAs (Si-1, Si-2 and Si-3) as encompassed by the present invention. As shown in the diagram, the sequences targeted by all three STAT3 DsiRNAs lie within a short 81-nt target region in the 3′UTR region of STAT3.

FIG. 4 shows the results of RT-qPCR quantification of the mRNA level of STAT1, STAT2, STAT3, STAT4, STAT5A and STAT5B in two lung cancer cell lines, H2170 and PC9. 50 nM of each customized STAT3 DsiRNAs was used, and JetPrime Transfection reagent was used according to manufacturer's protocol. Total RNA was harvested 48 hours post-transfection. GAPDH was used as endogenous control. The results show that all three STAT3 DsiRNAs demonstrated enhanced specificity in the knockdown of STAT3 mRNA.

FIG. 5 shows the results of Western blot validation of the protein level of Stat 1, Stat2, Stat3, Stat4, StatS and Stat6 in three different cancer cell lines (A549, PC9 and PC9-GR). Increasing concentrations of STAT3-DsiRNA3 (10, 20, 50 nM) per transfection were used for each cell line. Total protein was harvested 48 hours post-transfection with lysis buffer supplemented with protease and phosphatase inhibitors. β-actin was used as loading control. SDS-PAGE was performed with standard protocol (n=2). The results show that the efficacy and specificity of STAT3-DsiRNA3 were confirmed in all three cell lines, with strong inhibition of STAT33 protein but not of other STAT isoforms.

FIG. 6 shows results of functional validation of STAT3 knockdown using STAT3-DsiRNA3. Increasing concentrations of STAT3 DsiRNA3 (10, 20, 50 nM) per transfection were used for each cell line. A549, PC9 and PC9GR cells (5,000 cells) are grown in 0.36% soft agar 24-hour post-transfection, and anchorage-independent growth was quantified at assay endpoint. MTT solution was used to stain the viable colonies. Data was presented as mean±SD (n=3). The results show that cell lines with strong STAT3 activity (A549 and PC-9GR) formed large colonies, and that the STAT3-DsiRNA3 was able to reduce colony formation in a dose-dependent manner in all three cell lines.

FIG. 7 shows results of Western blot characterization of the protein level of hTfR1 and hTfR2 in normal lung epithelial NL20 and 11 lung cancer cell lines. Total protein was harvested 48 hours post-transfection with lysis buffer supplemented with protease and phosphatase inhibitors. β-actin was used as loading control. SDS-PAGE was performed with standard protocol (n=2). The results show that hTfRs, particularly hTfR1, are expressed in 10 out of 11 lung cancer cell lines, and that the majority of the 11 cancer cell lines have higher TfR1 than the normal lung epithelial cell line NL20.

FIG. 8 shows results of RT-qPCR quantification of the mRNA level of STAT3 and B2M in rat hepatoctye cell lines. 500 nM or 1000 nM of aptamers were used. Total RNA was harvested 48 hours post-transfection. B2M was used as endogenous control. The results show that both the control and STAT3 aptamers did not affect B2M gene (endogenous control), while a 55% knockdown of STAT3 mRNA was observed when using liaM STAT3-hTfR aptamer.

FIG. 9 shows results of Western blot validation of the protein level of Stat1 and Stat3 in cancer cells. 500nM or 1000nM of aptamers were used. Total protein was harvested 48 hours post-transfection with lysis buffer supplemented with protease and phosphatase inhibitors. β-actin was used as loading control. SDS-PAGE was performed with standard protocol (n=2). FIG. 9A H2170 and A549 cells were also transfected with 200 nM STAT3-hTfR aptamer using JetPrime Transfection Reagent as a positive control. FIG. 9B phosphorylated Stat3 (Y705 and 5727) were immunoblotted to investigate the efficacy of STAT3-hTfR aptamer in inhibiting Stat3 activities. The result showed that STAT3-hTfR aptamer had no effect on the hTfR-negative A549 cells, while reduction of Stat3 was observed in H2170 cell line. In addition, a clear suppression of phosphorylated Stat3 was observed in HCC827, but not so in PC9 cells, indicating that the aptamer has a higher selectivity towards hTfR1 than hTfR2. In addition, the results show that STAT3-DsiRNA-hTfR aptamer strongly abrogated the phosphorylation of STAT3 at residue Y705, with marginal effect on residue S727.

FIG. 10 shows the results of aligning the short 81-nt target region in the 3′ UTR region of STAT3 with the homo sapiens genome using Blastn. The results show that this 81-nt target region is common to various STAT3 variants.

FIG. 11 shows the results of RT-qPCR quantification of the mRNA level of STAT1, STAT2, STAT3 and STAT4 in A549 lung cancer cell line. 25, 50, or 100 nM of STAT3-20 ASO (FIG. 11A) or customized STAT3-DsiRNA3 (FIG. 11B) were used, and JetPrime Transfection reagent was used according to manufacturer's protocol. Total RNA was harvested 24 hours post-transfection. GAPDH was used as endogenous control. The results show that STAT3-DsiRNA3 achieved a more robust and specific knockdown of STAT3 as compared to STAT3-ASO.

FIG. 12 shows the results of RT-qPCR quantification of the knockdown efficiency of scrambled ASO (ASO Scr), AZD9150 (ASO 50 nM), scrambled DsiRNA (DsiRNA Scr), and STAT3 DsiRNA3 (DsiRNA 50 nM) over 6 days post-transfection (1D, 2D, 3D and 6D) in A549 lung cancer cell line. The mRNA levels of STAT1 (FIG. 12A), STAT2 (FIG. 12B), STAT3 (FIG. 12C) and STAT4 (FIG. 12D) were shown. 50 nM of STAT3-ASO or customized STAT3-DsiRNA3 were used, and JetPrime Transfection reagent was used according to manufacturer's protocol. Total RNA was harvested at the indicated time point. GAPDH was used as endogenous control. The results show that STAT3-DsiRNA3 demonstrated a superior specificity and potency than STATS-ASO starting from Day 1, and the strong knockdown efficacy could be sustained up to Day 6.

FIG. 13 shows the results of Seahorse XF Cell Mito Stress Test that measures the mitochondria functions, particularly the oxidative phosphorylation (OXPHOS) capacity. This assay provides measurements of the basal respiration and maximal respiration capacity of the mitochondria under different conditions. The effects of scrambled DsiRNA (Scr), and STAT3 DsiRNA3 (STAT3) at 50 nM for HCC827 (FIG. 13A) and Calu-1 (FIG. 13B) cell lines are presented. The results show that STAT3 DsiRNA has marginal effects in affecting mitochondrial OXPHOS in both cell types.

FIG. 14A shows results of Western blot characterization of the protein level of hTfR1 and hTfR2 in 14 patient-derived tumours extracted from xenotransplantion of hepatocellular carcinoma tumours (HCC-PDX) in animals. Total protein was harvested 48 hours post-transfection with lysis buffer supplemented with protease and phosphatase inhibitors. SDS-PAGE was performed with standard protocol. The results show that hTfR1 is expressed highly in 5 out of 14 HCC-PDX tumours, and moderately in 3/14 tumours. HCC10-0505 with a high TfR1 expression is selected for subsequent analysis. FIG. 14B shows results of Western blot analysis of the protein level of Stat3 (total and phosphorylated), the downstream kinase ERK1/2 and Akt, and the cell cycle markers (cyclin Bl, CDC25C, Cdc2 and Rb1) in HCC10-0505. 75 μl and 150 μl aptamers were administered twice weekly. Tumours are harvested 1-week post treatment for SDS-PAGE. Data shows a reduction of Stat3 tyrosine phosphorylation at both doses of the STAT3 DsiRNA. FIG. 14C show representative images of immunochemical staining for cleaved PARP (apoptotic marker) and p-Histone H3 (cell proliferation marker) in HCC10-0505 tumours post-treatment. The results show marginally changes in cleaved PARP, likely due to the short duration of aptamer treatment, but a clear reduction of p-Histone H3 marker is observed.

FIG. 15 shows effect of STAT3 DsiRNAs packaged in RBCEV in A549 cancer cell lines. FIG. 15A shows the results of RT-qPCR quantification of the mRNA level of STAT1, STAT2, STAT3 and STAT4 in A549 lung cancer cell line. 100 nM of control (si-NC) and STAT3-DsiRNA (si-STAT3) were treated for 6 h, 24, and 48 h. Total RNA was harvested 24 hours post-transfection. GAPDH was used as endogenous control. The results show that si-STAT3 achieved a more robust and specific knockdown of STAT3 as compared to si-NC. FIG. 15B shows results of Western blot validation of the protein level of Stat1 and Stat3 in cancer cells. 100 nM were used. Total protein was harvested 48 hours post-transfection with lysis buffer supplemented with protease and phosphatase inhibitors. β-actin was used as loading control. SDS-PAGE was performed with standard protocol (n=3) to investigate the specificity and efficacy of STAT3-hTfR aptamer in inhibiting Stat3 activities. The result showed that STATS RBCEV had no effect on the Stat1, but a clear suppression of phosphorylated Stat3 was observed, reiterating that the STATS-RBCEV retained its specificity towards Stat3.

### DEFINITIONS

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which, in certain cases, are metabolized in a manner similar to the reference nucleotides.

As used herein, the term “nucleoside” refers to a glycosylamine comprising a nucleobase and a sugar. Nucleosides includes, but are not limited to, natural nucleosides, abasic nucleosides, modified nucleosides, and nucleosides having mimetic bases and/or sugar groups. As used herein, the term “natural nucleoside” or “unmodified nucleoside” means a nucleoside comprising a natural nucleobase and a natural sugar. Natural nucleosides include RNA and DNA nucleosides. As used herein, the term “nucleobase” refers to the base portion of a nucleoside or nucleotide. A nucleobase may comprise any atom or group of atoms capable of hydrogen bonding to a base of another nucleic acid. As used herein, the term “natural nucleobase” refers to a nucleobase that is unmodified from its naturally occurring form in RNA or DNA. Examples of “natural nucleobases” include the purine nucleobases adenine (A) and guanine (G), and the pyrimidine nucleobases thymine (T), cytosine (C) and uracil (U). In addition to the “natural nucleobases”, many modified nucleobases or nucleobase mimetics known to those skilled in the art are amenable with the compounds described herein. The terms “modified nucleobase” and “nucleobase mimetic” can overlap but generally a “modified nucleobase” refers to a nucleobase that is fairly similar in structure to the parent nucleobase, such as for example a 7-deaza purine, a 5-methyl cytosine, or a G-clamp, whereas a “nucleobase mimetic” would include more complicated structures, such as for example a tricyclic phenoxazine nucleobase mimetic.

As used herein, the term “nucleotide” refers to a nucleoside having a phosphate group covalently linked to the sugar. Nucleotides may be modified with any of a variety of substituents.

As used herein, the term “oligonucleotide” refers to an oligomeric compound comprising a plurality of linked nucleotides. As used herein, the term “oligomeric compound” refers to a polymeric structure comprising two or more sub-structures and capable of hybridizing to a region of a nucleic acid molecule. In some examples, oligonucleotides can be introduced in the form of single-stranded, double-stranded, circular, branched or hairpins and can contain structural elements such as internal or terminal bulges or loops. Double-stranded oligonucleotides can be formed by two oligonucleotide strands hybridized together, or a single oligonucleotide strand with sufficient self-complementarity to allow for hybridization and formation of a fully or partially double-stranded compound.

As used herein, the term “target region” refers to a contiguous portion of the nucleotide sequence of an mRNA molecule formed during the transcription of an STAT3 gene. In one example, the target region is within the three prime untranslated region (3′-UTR) of STAT3 mRNA. In one specific example, the target region of STAT3 mRNA has the sequence of

(SEQ ID NO: 1)
AGGUCAAACCCUUAAGACAUCUGAAGCUGCAACCUGGCCUUUGGUGUUG
AAAUAGGAAGGUUUAAGGAGAAUCUAAGCAUU.

A target region may contain one or more target segments. Multiple target segments within a target region may be overlapping. Alternatively, they may be non-overlapping. In some examples, target segments within a target region are separated by a number of nucleotides that is about, or is no more than about 50, 45, 40, 35, 30, 25, 20, 15, 10, 5, 2 or 1 nucleotides on the target nucleic acid, or is a range defined by any two of the preceding values. In some examples, target segments are contiguous.

As used herein, the term “complementary” refers to the capacity of an oligomeric compound to hybridize to another oligomeric compound or nucleic acid through nucleobase complementarity. In some examples, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, disclosed herein are antisense compounds that can comprise up to about 20% nucleotides that are mismatched (i.e., are not nucleobase complementary to the corresponding nucleotides of the target). Preferably the antisense compounds contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% nucleobase complementary to a target nucleic acid.

As used herein, the term “percent complementarity” refers to the number of nucleobases of an oligomeric compound that have nucleobase complementarity with a corresponding nucleobase of another oligomeric compound or nucleic acid, divided by the total length (number of nucleobases) of the oligomeric compound. For example, an oligomeric compound A of 15-nucleotide long has 12 nucleobase complementarity with oligomeric compound B, then the percent complementary of oligomeric compound A to oligomeric compound B is 12/15=80%. Percent complementarity of an antisense compound with a region of a target nucleic acid can be determined routinely using BLAST programs.

As used herein, the term “hybridization” means the pairing of complementary oligomeric compounds (e.g. an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases. For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

Two sequences may be complementary and hybridize to each other under moderately stringent, or preferably stringent, conditions. Hybridization to desired sequences under moderately stringent conditions or under stringent conditions can be performed by methods known in the art. Hybridization conditions can also be modified in accordance with known methods depending on the sequence of interest.

As used herein, “mismatch” or “non-complementary nucleobase” refers to the case when a nucleobase of a first nucleic acid is not capable of pairing with the corresponding nucleobase of a second or target nucleic acid.

As used herein, the term “loop” refers to a structure formed by a single strand of a nucleic acid, in which complementary regions that flank a particular single stranded nucleotide region hybridize in a way that the single stranded nucleotide region between the complementary regions is excluded from duplex formation or Watson-Crick base pairing. A loop is a single stranded nucleotide region of any length. Examples of loops include the unpaired nucleotides present in such structures as hairpins, stem loops, or extended loops.

As used herein, a “double-stranded nucleic acid” or “dsNA” is a molecule comprising two oligonucleotide strands which form a duplex. A dsNA may contain ribonucleotides, deoxyribonucleotides, modified nucleotides, and combinations thereof. The dsNA molecules can be either a dicer substrate or a non-dicer substrate. Dicer, also known as endoribonuclease Dicer or helicase with RNase motif, is an enzyme that in humans is encoded by the DICER1 gene. Dicer is an endoribonuclease in the RNase III family that cleaves a dsRNA or dsRNA-containing molecule, e.g., double-stranded RNA (dsRNA) or pre-microRNA (miRNA), into double-stranded nucleic acid fragments about 19-25 nucleotides long, usually with a two-base overhang on the 3′ end. With respect to the dsNAs as disclosed herein, the duplex formed by a dsRNA region of a dsNA is recognized by Dicer and is a Dicer substrate on at least one strand of the duplex. Dicer catalyzes the first step in the RNA interference pathway, which consequently results in the degradation of a target RNA. In examples where the dsNA molecule is a dicer substrate, the dsNA molecule is referred to as a dicer substrate NA (dsiNA). In examples where the dsiNA comprises a plurality of RNA, it is referred to as a dicer substrate siRNA (DsiRNA). DsiRNA molecules comprise both DNA and RNA molecules. DsiRNA is a subset of DsiNA which is a subset of dsNA. Therefore the term dsNA is inclusive of dsiNA, DNA duplex, DNA/RNA duplex, RNA duplex and DsiRNA. In some examples, the dsNAs comprise an RNA duplex in a region that is capable of functioning as a Dicer substrate siRNA (DsiRNA), and a single stranded region, which is located at a position 5′ of the projected Dicer cleavage site of the second strand of the DsiRNA/DNA agent. In another example, the dsNA comprises an RNA duplex in a region that is capable of functioning as a Dicer substrate siRNA (DsiRNA) and a single stranded region, which is located at a position 3′ of the projected Dicer cleavage site of the first strand of the DsiRNA/DNA agent. In another example, the dsNA comprises an RNA duplex that is a Dicer substrate siRNA (DsiRNA) and a single stranded region comprising at least one modified nucleotide and/or phosphate backbone modification, which is located at a position 3′ of the projected Dicer cleavage site of the second strand of the DsiRNA/DNA agent. In another example, the dsNA comprises an RNA duplex that is a Dicer substrate siRNA (DsiRNA) and a single stranded region comprising at least one modified nucleotide and/or phosphate backbone modification, which is located at a position 5′ of the projected Dicer cleavage site of the first strand of the DsiRNA/DNA agent.

DsiRNAs can possess certain advantages as compared to inhibitory nucleic acids that are not dicer substrates (“non-DsiRNAs”). Such advantages include, but are not limited to, enhanced duration of effect of a DsiRNA relative to a non-DsiRNA, as well as enhanced inhibitory activity of a DsiRNA as compared to a non-DsiRNA (e.g., a 19-23mer siRNA) when each inhibitory nucleic acid is suitably formulated and assessed for inhibitory activity in a mammalian cell at the same concentration.

As used herein, the term “antisense compound” refers to an oligomeric compound that is at least partially complementary to a target nucleic acid molecule to which it hybridizes, and the term “antisense” should be construed accordingly. In some examples, an antisense compound modulates, in particular decreases, expression of a target nucleic acid. Antisense compounds include, but are not limited to, compounds that are oligonucleotides, oligonucleosides, oligonucleotide analogs, oligonucleotide mimetics, and chimeric combinations of these. The term “antisense oligonucleotide” refers to an antisense compound that is an oligonucleotide, and the term “antisense strand” refers to an antisense compound that is a single stranded nucleic acid molecule. As used herein, the term “sense strand” refers to a single stranded nucleic acid molecule which has a sequence complementary to that of an antisense strand.

As used herein, the term “modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

As used herein, the term “modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

As used herein, the term “modified oligonucleotide” means an oligonucleotide comprising a modified internucleoside linkage, a modified sugar, and/or a modified nucleobase.

As used herein, the term “modified internucleoside linkage” refers to any linkage between nucleosides other than a naturally occurring internucleoside linkage. Modified internucleoside linkages, compared to natural phosphodiester linkages, can be used to alter, typically increase, nuclease resistance of the antisense compound.

As used herein, the term “modified sugar” refers to a substitution or change from a natural sugar.

As used herein, the term “naturally occurring internucleoside linkage” means a 3′ to 5′ phosphodiester linkage.

As used herein, the term “natural sugar moiety” means a sugar found in DNA (2′-H) or RNA (2′-OH).

As used herein, the term “siRNA” refers to a double stranded nucleic acid in which each strand comprises RNA, RNA analog(s) or RNA and DNA. The siRNA typically comprises between 19 and 23 nucleotides, or comprises 21 nucleotides in some specific examples. The siRNA typically has 2 base pair (bp) overhang on the 3′ ends of each strand, such that the duplex region in the siRNA comprises 17 to 21 nucleotide base pairs, or 19 nucleotide base pairs. Typically, the antisense strand of the siRNA is sufficiently complementary to the target sequence. siRNAs trigger RNA interference without Dicer cleavage.

As used herein, the term “duplex region” refers to the region in which two complementary or substantially complementary oligonucleotides form base pairs with one another, either by Watson-Crick base pairing or any other manner that allows for a duplex between oligonucleotide strands that are complementary or substantially complementary. For example, an oligonucleotide strand having 21 nucleotides can base pair with another oligonucleotide of 21 nucleotides, yet only 19 nucleotides on each strand are complementary or substantially complementary, such that the “duplex region” consists of 19 base pairs. The remaining base pairs may, for example, exist as 5′ and 3′ overhangs. Further, within the duplex region, 100% complementarity is not required, substantial complementarity is allowable within a duplex region. Substantial complementarity refers to complementarity between the strands such that they are capable of annealing under biological conditions. Techniques to empirically determine if two strands are capable of annealing under biological conditions are well known in the art. The following is a schematic diagram of a duplex siRNA molecule with 3′ overhangs in both oligonucleotide strands and a duplex region of 19 nucleotide base pairs.

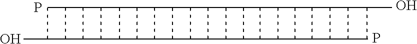

As used herein, the term “targeting” or “targeted to” refers to the association of a compound to a particular target nucleic acid molecule or a particular region of nucleotides within a target nucleic acid molecule. An antisense compound targets a target nucleic acid if it is sufficiently complementary to the target nucleic acid to allow hybridization under physiological conditions.

As used herein, the term “aptamer” refers to oligonucleotide or peptide molecules that can bind to a specific target molecule. Aptamers are usually created by selecting them from a large random sequence pool, but natural aptamers also exist in riboswitches. Aptamers can usually be classified as nucleic acid aptamers (including aptamers consisting of DNAs, RNAs, analogues thereof, and combinations thereof) and peptide aptamers.

As used herein, the term “nanomaterial” refers to any material the size of which is measured in nanometers, e.g., a particle with at least one dimension less than about 100 nm.

As used herein, the term “nanoparticle” refers to a microscopic particle, the size of which is measured in nanometers, e.g., a particle with at least one dimension less than about 100 nm. Nanoparticles can be used in therapeutic applications as drug carriers that, when conjugated to an aptamer component as disclosed herein, deliver the nucleic acid or dsNA component and optionally other therapeutic agents, cytotoxic or anti-cancer agents known in the art.

As used herein, the term “nanomicelle” refers to nanoscopic globular structures that consist of exterior hydrophilic polar heads and interior hydrophobic fatty acyl chain. Nanomicelles are typically spherical, but can sometimes take other shapes, such as cylinders and ellipsoids.

As used herein, the term “liposome” refers to a spherical vesicle having one or more phospholipid bilayers.

As used herein, the term “dendrimer” refers to highly branched, star-shaped macromolecules with nanometer-scale dimensions. Dendrimers are typically defined by three components: a central core, an interior dendritic structure (the branches), and an exterior surface with functional surface groups.

As used herein, the term “inert antibody” refers to an antibody that specifically bind its target antigen but does not modulate (e.g., decrease/inhibit or activate/induce) antigen function. It would be appreciated that methods of preparing inert antibodies specific for a particular target antigen and/or a particular cell type are well known in the art.

As used herein, the term “poloxamer” refers to nonionic triblock copolymer composed of a central hydrophobic chain of polyoxypropylene (poly(propylene oxide)) flanked by two hydrophilic chains of polyoxyethylene (poly(ethylene oxide)). Various gel formulations of poloxamers can be prepared using methods known in the art.

As used herein, the term “hydrocarbon” refers to a compound containing only carbon and hydrogen. A hydrocarbon can be acyclic or cyclic, and/or can be linear or branched. A hydrocarbon can include rings, ring systems, aromatic rings, and aromatic ring systems, which contain only carbon and hydrogen. Hydrocarbons include saturated hydrocarbons, unsaturated hydrocarbons, and aromatic hydrocarbons. Saturated hydrocarbons are the simplest form of hydrocarbons. They are composed entirely of single bonds and are saturated with hydrogen.

The formula for acyclic saturated hydrocarbons (i.e., alkanes) is CₙH₂ₙ₊₂. The most general form of saturated hydrocarbons is CₙH₂ₙ₊₂(i1-r), where r is the number of rings. Saturated hydrocarbons with exactly one ring are called cycloalkanes. Unsaturated hydrocarbons have one or more double or triple bonds between carbon atoms. Those with double bond are called alkenes. Unsaturated hydrocarbons with one double bond have the formula CₙHₙ₂ (assuming non-cyclic structures). Unsaturated hydrocarbons containing triple bonds are called alkyne. Unsaturated hydrocarbons with one triple bond have the formula CₙH₂ₙ₋₂. Aromatic hydrocarbons, also known as arenes, are hydrocarbons that have at least one aromatic ring.

The term “sugar modification” or “modified sugar” as used interchangeably herein refers to a sugar moiety that is not a ribofuranosyl as found in naturally occurring RNA or a deoxyribofuranosyl as found in naturally occurring DNA. Modified sugar moieties can be used to alter, typically increase, the affinity of the antisense compound for its target and/or increase nuclease resistance. A “modified sugar” includes but is not limited to a substituted sugar, a bicyclic or tricyclic sugar, or a sugar surrogate. As used herein, “substituted sugar moiety” means a furanosyl comprising at least one substituent group that differs from that of a naturally occurring sugar moiety. Substituted sugars include, but are not limited to, furanosyls comprising substituents at the 2′-position, the 3′-position, the 5′-position and/or the 4′-position. As used herein, “2′-substituted sugar” means a furanosyl comprising a substituent at the 2′-position other than H or OH. Unless otherwise indicated, a 2′-substituted sugar is not a bicyclic sugar (i.e., the 2∝-substituent of a 2′-substituted sugar moiety does not form a bridge to another atom of the furanosyl ring). Examples of sugar substituents suitable for the 2′-position include, but are not limited to: 2′-O-methyl, 2′-O-methoxyethyl, and 2′-fluoro. In some examples, sugar substituents at the 2′ position is selected from allyl, amino, azido, thio, O-allyl, O—C₁-C₁₀ alkyl, O—C₁-C₁-C₁₀ substituted alkyl; O—C₁₀ alkoxy; O-C₁-C₁₀ substituted alkoxy, OCF₃, O(CH₂)₂SCH₃, O(CH₂)₂—O-N(Rm)(Rn), and O—CH₂—C(═O)—N(Rm)(Rn), where each Rm and Rn is, independently H or substituted or unsubstituted C₁-C₁₀ alkyl.

As used herein, “bicyclic sugar” means a modified sugar comprising a 4 to 7 membered ring (including but not limited to a furanosyl) comprising a bridge connecting two atoms of the 4 to 7 membered ring to form a second ring, resulting in a bicyclic structure. In some examples, the 4 to 7 membered ring is a sugar ring. In some examples, the 4 to 7 membered ring is a furanosyl. In some of such examples, the bridge connects the 2′-carbon and the 4′-carbon of the furanosyl.

As used herein, the term “bicyclic nucleoside” or “BNA” refers to a nucleoside wherein the furanose portion of the nucleoside includes a bridge connecting two atoms on the furanose ring, thereby forming a bicyclic ring system. BNAs include, but are not limited to, α-L-LNA, β-D-LNA, ENA, Oxyamino BNA (2′-O—N(CH₃)—CH₂-4′) and Aminooxy BNA (2′-N(CH₃)—O—CH₂-4′).

Representative structures of BNA's include but are not limited to:

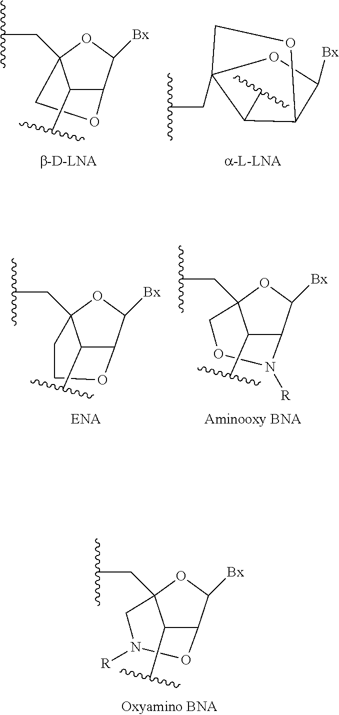

As used herein, the term “4′ to 2′ bicyclic nucleoside” refers to a BNA wherein the bridge connecting two atoms of the furanose ring bridges the 4′ carbon atom and the 2′ carbon atom of the furanose ring, thereby forming a bicyclic ring system.

As used herein, a “locked nucleic acid” or “LNA” refers to a nucleotide modified such that the 2′-hydroxyl group of the ribosyl sugar ring is linked to the 4′ carbon atom of the sugar ring via a methylene groups, thereby forming a 2′-C,4′-C-oxymethylene linkage. LNAs include, but are not limited to, α-L-LNA, and β-D-LNA.

As used herein, the term “sugar surrogate” means a structure that does not comprise a furanosyl and that is capable of replacing the naturally occurring sugar of a nucleoside, such that the resulting nucleoside is capable of (1) incorporation into an oligonucleotide and (2) hybridization to a complementary nucleoside. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholino, modified morpholinos, cyclohexenyls and cyclohexitols.

As used herein, the term “pharmaceutically acceptable salts” refers to salts of active compounds that retain the desired biological activity of the active compound and do not impart undesired toxicological effects thereto. Sodium salts of antisense oligonucleotides are useful and are well accepted for therapeutic administration to humans

As used herein, the term “prodrug” refers to a therapeutic agent that is prepared in an inactive or less active form that is converted to an active form (i.e., drug) within the body or cells thereof by the action of endogenous enzymes, chemicals, and/or conditions. Prodrugs can also include antisense compounds wherein one or both ends comprise nucleobases that are cleaved (e.g., by incorporating phosphodiester backbone linkages at the ends) to produce the active compound. Examples of prodrugs include but are not limited to carboxylicacidesters prodrugs (in which the hydroxyl group located at the side chain of the nucleoside is esterified with organic acid), monophosphate prodrugs, and amide-type prodrugs.

As used herein, the term “treatment” refers to administering a composition as disclosed to effect an alteration or improvement of the disease or condition. Prevention, amelioration, and/or treatment may require administration of multiple doses at regular intervals, or prior to onset of the disease or condition to alter the course of the disease or condition. Moreover, a single agent may be used in a single individual for each prevention, amelioration, and treatment of a condition or disease sequentially, or concurrently.

As used herein, the term “pharmaceutical agent” refers to a substance which provides a therapeutic benefit when administered to a subject.

As used herein, the term “therapeutically effective amount” refers to an amount of a pharmaceutical agent that provides a therapeutic benefit to an animal.

As used herein, “administering” means providing a pharmaceutical agent to an animal, and includes, but is not limited to administering by a medical professional and self-administering.

As used herein, the term “pharmaceutical composition” refers to a mixture of substances suitable for administering to an individual. For example, a pharmaceutical composition may comprise an oligonucleotide and a sterile aqueous solution.

As used herein, the term “animal” refers to a human or non-human animal, including, but not limited to, mice, rats, rabbits, dogs, cats, pigs, and non-human primates, including, but not limited to, monkeys and chimpanzees.

### DETAILED DESCRIPTION OF THE PRESENT INVENTION

The inventors of the present application have uncovered that the three prime untranslated region (3′-UTR) of STAT3 mRNA contains a targeting region. It has been surprisingly found that compounds, in particular oligonucleotides, which target this targeting region, can effectively inhibit the expression of STAT3 with high specificity. The inhibition of STAT3 expression can effectively reduce colony formation in cancer cell lines. Thus, compounds which target this targeting region identified by the inventors of the present application serve as promising therapeutic candidates for tumors/cancers that are associated with STAT3 expression.

Thus, in one aspect, there is provided a nucleic acid comprising an oligonucleotide strand of 15-80 nucleotides in length, wherein said oligonucleotide strand is at least 80% complementary to a target STAT3 mRNA sequence along at least 15 nucleotides of said oligonucleotide strand, wherein the target STAT3 mRNA sequence has the sequence of

(SEQ ID NO: 1)
AGGUCAAACCCUUAAGACAUCUGAAGCUGCAACCUGGCCUUUGGUGUUG
AAAUAGGAAGGUUUAAGGAGAAUCUAAGCAUU.

In some examples, the oligonucleotide strand is about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or more nucleotides in length. Further, the length of the oligonucleotide strand may be defined by a range of any two values as provided above or any two values in between. In some specific examples, the oligonucleotide strand is of about 20 to nucleotides, or about 21 to 29 nucleotides, or about 22 to 28 nucleotides, or about 23 to 27 nucleotides in length. In one specific example, the oligonucleotide strand is of about 27 nucleotides in length.

In some examples, the oligonucleotide strand is at least 80%, 85%, 90%, 95%, 98%, 99% or 100% complementary to the sequence of SEQ ID NO: 1 along at least 15, 20, 25, 30, 40, 45, 50, 55, 60, 65, 70, 75 or 80 nucleotides of the oligonucleotide strand. The oligonucleotide strand is at least 80% complementary to a target STAT3 mRNA sequence (SEQ ID NO: 1) along at least 15 nucleotides of said oligonucleotide strand, means that said oligonucleotide strand has at least 12 nucleotides complementary to SEQ ID NO: 1. The at least 12 nucleotides that are complementary to SEQ ID NO: 1 can be contiguous (i.e. with no unmatched or mismatched nucleotides in between) or non-contiguous. When the at least 12 nucleotides that are complementary to SEQ ID NO: 1 are non-contiguous, it means the remaining non-complementary nucleotides in the oligonucleotide strand may be clustered or interspersed with the complementary nucleotides and need not be contiguous to each other or to complementary nucleotides. In some examples in which the at least 12 nucleotides that are complementary to SEQ ID NO: 1 are non-contiguous, there are at most 1, 2, 3, 4, 5, 6, 7, 8, 9, 10, 11, 12, 13, 14, 15 or 16 unmatched or mismatched nucleotides clustered or interspersed with the complementary nucleotides. For example, an oligonucleotide strand which is 15 nucleotides in length having three non-complementary nucleotides which are flanked by two regions (e.g. of six nucleotides long each) of complete complementarity with the sequence of SEQ ID NO: 1 would have 80% overall complementarity with the SEQ ID NO: 1 and would thus fall within the scope of the present invention. Similarly, an oligonucleotide strand which is 80 nucleotides in length having 16 non-complementary nucleotides which are interspersed with the nucleotides of complete complementarity with the sequence of SEQ ID NO: 1 would have 80% overall complementarity with the SEQ ID NO: 1 and would thus also fall within the scope of the present invention. In some examples, the non-complementary nucleotides are in a contiguous cluster of at least 2, 3, 4, 5, 6, 7, 8, 9, 10, 11, 12, 13, 14, 15 or 16 nucleotides long.

In some examples, the nucleic acid comprising the oligonucleotide strand as disclosed herein is an antisense oligonucleotide (ASO) or a short hairpin RNA (shRNA).

In another aspect, there is provided a double stranded nucleic acid (dsNA) for inhibiting expression of STAT3, comprising a sense strand and an antisense strand forming a double stranded region, wherein the antisense strand is at least 80% complementary to a target STAT3 mRNA sequence along at least 15 nucleotides of said antisense strand, wherein the target STAT3 mRNA sequence has the sequence of SEQ ID NO: 1; and wherein the sense strand is at least 80% complementary to the sequence AAUGCUUAGAUUCUCCUUAAACCUUCCUAUUUCAACACCAAAGGCCAGGUUGC AGCUUCAGAUGUCUUAAGGGUUUGACCUGA (SEQ ID NO: 2), along at least 15 nucleotides of said sense strand.

In some examples, the antisense strand is about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or more nucleotides in length. Further, the length of the antisense strand may be defined by a range of any two values as provided above or any two values in between. In some specific examples, the antisense strand is of about 20 to 30 nucleotides, or about 21 to 29 nucleotides, or about 22 to 28 nucleotides, or about 23 to 27 nucleotides, or about 24 to 26 nucleotides in length. In one specific example, the antisense strand is of about 25 nucleotides in length.

In some examples, the antisense strand is at least 80%, 85%, 90%, 95%, 98%, 99% or 100% complementary to the sequence of SEQ ID NO: 1 along at least 15, 20, 25, 30, 35, 40, 45, 50, 55, 60, 65, 70, 75 or 80 nucleotides of the antisense strand. The antisense strand is at least 80% complementary to a target STAT3 mRNA sequence (SEQ ID NO: 1) along at least 15 nucleotides of said antisense strand, means that said antisense strand has at least 12 nucleotides complementary to SEQ ID NO: 1. The at least 12 nucleotides that are complementary to SEQ ID NO: 1 can be contiguous (i.e. with no unmatched or mismatched nucleotides in between) or non-contiguous. When the at least 12 nucleotides that are complementary to SEQ ID NO: 1 are non-contiguous, it means the remaining non-complementary nucleotides in the antisense strand may be clustered or interspersed with the complementary nucleotides and need not be contiguous to each other or to complementary nucleotides. In some examples in which the at least 12 nucleotides that are complementary to SEQ ID NO: 1 are non-contiguous, there are at most 1, 2, 3, 4, 5, 6, 7, 8, 9, 10, 11, 12, 13, 14, 15 or 16 unmatched or mismatched nucleotides clustered or interspersed with the complementary nucleotides. For example, an antisense strand which is 15 nucleotides in length having three non-complementary nucleotides which are flanked by two regions (e.g. of six nucleotides long each) of complete complementarity with the sequence of SEQ ID NO: 1 would have 80% overall complementarity with the SEQ ID NO: 1 and would thus fall within the scope of the antisense strand as disclosed herein. Similarly, an antisense strand which is 80 nucleotides in length having 16 non-complementary nucleotides which are interspersed with the nucleotides of complete complementarity with the sequence of SEQ ID NO: 1 would have 80% overall complementarity with the SEQ ID NO: 1 and would thus also fall within the scope of the antisense strand as disclosed herein. In some examples, the non-complementary nucleotides are in a contiguous cluster of at least 2, 3, 4, 5, 6, 7, 8, 9, 10, 11, 12, 13, 14, 15 or 16 nucleotides long.

In some examples, the sense strand is about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 76, 77, 78, 79, 80, 81, 82 or more nucleotides in length. Further, the length of the sense strand may be defined by a range of any two values as provided above or any two values in between. In some specific examples, the sense strand is of about 20 to 30 nucleotides, or about 21 to 29 nucleotides, or about 22 to 28 nucleotides, or about 23 to 27 nucleotides in length. In one specific example, the sense strand is of about 27 nucleotides in length.

In some examples, the sense strand is at least 80%, 85%, 90%, 95%, 98%, 99% or 100% complementary to the sequence of SEQ ID NO: 2 along at least 15, 20, 25, 30, 35, 40, 50, 55, 60, 65, 70, 75 or 80 nucleotides of the sense strand. The sense strand is at least 80% complementary to the sequence of SEQ ID NO: 2 along at least 15 nucleotides of said sense strand, means that said sense strand has at least 12 nucleotides complementary to SEQ ID NO: 2. The at least 12 nucleotides that are complementary to SEQ ID NO: 2 can be contiguous (i.e. with no unmatched or mismatched nucleotides in between) or non-contiguous. When the at least 12 nucleotides that are complementary to SEQ ID NO: 2 are non-contiguous, it means the remaining non-complementary nucleotides in the sense strand may be clustered or interspersed with the complementary nucleotides and need not be contiguous to each other or to complementary nucleotides. In some examples in which the at least 12 nucleotides that are complementary to SEQ ID NO: 2 are non-contiguous, there are at most 1, 2, 3, 4, 5, 6, 7, 8, 9, 11, 12, 13, 14, 15 or 16 unmatched or mismatched nucleotides clustered or interspersed with the complementary nucleotides. For example, a sense strand which is 15 nucleotides in length having three non-complementary nucleotides which are flanked by two regions (e.g. of six nucleotides long each) of complete complementarity with the sequence of SEQ ID NO: 2 would have 80% overall complementarity with the SEQ ID NO: 2 and would thus fall within the scope of the sense strand as disclosed herein. Similarly, a sense strand which is 80 nucleotides in length having 16 non-complementary nucleotides which are interspersed with the nucleotides of complete complementarity with the sequence of SEQ ID NO: 2 would have 80% overall complementarity with the SEQ ID NO: 2 and would thus also fall within the scope of the sense strand as disclosed herein. In some examples, the non-complementary nucleotides are in a contiguous cluster of at least 2, 3, 4, 5, 6, 7, 8, 9, 10, 11, 12, 13, 14, 15 or 16 nucleotides long.

In some specific examples, the antisense strand comprises at least 15, 16, 17, 18, 19, 21, 22, 23, 24 or 25 contiguous nucleotides that are at least 80%, 85%, 90%, 95%, 98%, 99% or 100% complementary to the following nucleotide sequences: 5′-AAGGUUUAAGGAGAAUCUAAGCAUU-3′ (SEQ ID NO: 3), 5′-AGGUCAAACCCUUAAGACAUCUGAA-3′ (SEQ ID NO: 5) or 5′-GGUCAAACCCUUAAGACAUCUGAAG-3′ (SEQ ID NO: 7). In some examples, the antisense strand comprises at least 15 contiguous nucleotides that are at least 80% complementary to the sequences of SEQ ID NO: 3, 5, or 7. In some other examples, the antisense strand comprises at least 15 contiguous nucleotides that are 100% complementary to the sequences of SEQ ID NO: 3, 5, or 7. In some other examples, the antisense strand comprises contiguous nucleotides that are 100% complementary to the sequences of SEQ ID NO: 3, 5, or 7. It is to be noted that the sequences of SEQ ID NOs 3, 5 and 7 are not distinguishing DNAs and RNAs, i.e. the nucleotides in the sequences of SEQ ID NOs 3, 5 and 7 can be deoxyribonucleotides, ribonucleotides, modified nucleotides, or combinations thereof.

In some specific examples, the sense strand comprises at least 15, 16, 17, 18, 19, 20, 21, 22, 23, 24, 25, 26 or 27 contiguous nucleotides that are at least 80%, 85%, 90%, 95%, 98%, 99% or 100% complementary to the following nucleotide sequences: 5′-

AAUGCUUAGAUUCUCCUUAAACCUUCC-3′ (SEQ ID NO: 4), 5′-UUCAGAUGUCUUAAGGGUUUGACCUGA-3′ (SEQ ID NO: 6) or 5′-CUUCAGAUGUCUUAAGGGUUUGACCUG-3′ (SEQ ID NO: 8). In some examples, the sense strand comprises at least 15 contiguous nucleotides that are at least 80% complementary to the sequences of SEQ ID NO: 4, 6, or 8. In some other examples, the sense strand comprises at least 15 contiguous nucleotides that are 100% complementary to the sequences of SEQ ID NO: 4, 6, or 8. In some other examples, the sense strand comprises 27 contiguous nucleotides that are 100% complementary to the sequences of SEQ ID NO: 4, 6, or 8. It is to be noted that the sequences of SEQ ID NOs 4, 6 and 8 are not distinguishing DNAs and RNAs, i.e. the nucleotides in the sequences of SEQ ID NOs 4, 6 and 8 can be deoxyribonucleotides, ribonucleotides, modified nucleotides, or combinations thereof.

Examples of dsNAs include but are not limited to double stranded ribonucleotides (RNAs), deoxyribonucleotides (DNAs), modified nucleotides, and combinations thereof. In one specific example, the dsNA is a small interfering RNA (siRNA). In another specific example, the dsNA is a dicer substrate siRNA (DsiRNA).

In some examples, the sense strand of the DsiRNA comprises at least 15, 16, 17, 18, 19, 20, 21, 22, 23, 24 or 25 contiguous nucleotides differing by 1, 2, or 3 nucleotides, or no more than 1, 2, or 3 nucleotides, from the following sequences: 5′-rArArGrGrUrUrUrArArGrGrArGrArArUrCrUrArArGrCrATT-3′ (SEQ ID NO: 9), 5′-rArGrGrUrCrArArArCrCrCrUrUrArArGrArCrArUrCrUrGAA-3′ (SEQ ID NO: 11) or 5′-rGrGrUrCrArArArCrCrCrUrUrArArGrArCrArUrCrUrGrAAG-3′ (SEQ ID NO: 13) (rA, rG, rU and rC refer to ribonucleotides). In some specific examples, the sense strand of the DsiRNA comprises at least 15 contiguous nucleotides differing by no more than 3 nucleotides from the sequence of SEQ ID NO: 9, SEQ ID NO: 11, or SEQ ID NO: 13. In some specific examples, the sense strand of the DsiRNA comprises the sequence of SEQ ID NO: 9, 11 or 13. In some examples, the antisense strand that is complementary to SEQ ID NO: 9 has the sequence 5′-rArArUrGrCrUrUrArGrArUrUrCrUrCrCrUrUrArArArCrCrUrUrCrC-3′ (SEQ ID NO: 10), the antisense strand that is complementary to SEQ ID NO: 11 has the sequence 5′-rUrUrCrArGrArUrGrUrCrUrUrArArGrGrGrUrUrUrGrArCrCrUrGrA-3′ (SEQ ID NO: 12), and the antisense strand that is complementary to SEQ ID NO: 13 has the sequence 5′-rCrUrUrCrArGrArUrGrUrCrUrUrArArGrGrGrUrUrUrGrArCrCrUrG-3′ (SEQ ID NO: 14). Thus, in some examples, the antisense strand of the DsiRNA comprises at least 15, 16, 17, 18, 19, 20, 21, 22, 23, 24, 25, 26 or 27 contiguous nucleotides differing by 1, 2, or 3 nucleotides, or no more than 1, 2, or 3 nucleotides, from the sequences of SEQ ID NO: 10, 12 or 14. In some specific examples, the antisense strand of the DsiRNA comprises at least 15 contiguous nucleotides differing by no more than 3 nucleotides from the sequence of SEQ ID NO: 10, SEQ ID NO: 12, or SEQ ID NO: 14. In some specific examples, the antisense strand of the DsiRNA comprises the sequence of SEQ ID NO: 10, 12 or 14.

When designing the dsNAs, a person skilled in the art can use the protocols available in the field to determine the appropriate lengths of the sense strand and the antisense strand of the dsNA. In some examples, the sense strand and/or the antisense strand is of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or more nucleotides in length. Further, the length of the sense and/or antisense strand may be defined by a range of any two values as provided above or any two values in between. In some specific examples, the sense strand is 15-80 nucleotides in length, and the antisense strand is 19-80 nucleotides in length. In some other specific examples, the sense strand is 15-35 nucleotides in length, and the antisense strand is 19-35 nucleotides in length.

The sense strand and the antisense strand of the dsNAs form a duplex region by hybridization. In some examples, the length of the duplex region is 15 to 30 base pairs, or 16 to 29 base pairs, or 17 to 28 base pairs, or 18 to 27 base pairs, or 19 to 26 base pairs, or 20 to base pairs, or 21 to 24 base pairs, or 15, 16, 17, 18, 19, 20, 21, 22, 23, 24, 25, 26, or 27 base pairs. In one specific example, the length of the duplex region is 25 base pairs.

In some examples, the antisense strand and the sense strand can be chemically linked outside their duplex region by chemical linking groups. Many suitable chemical linking groups are known in the art and can be used. Suitable groups will not block Dicer activity on the DsiRNA and will not interfere with the directed destruction of the RNA transcribed from the target gene. Alternatively, the antisense strand and the sense strand can be linked by an oligonucleotide such that a hairpin structure is produced upon annealing of the two strands. The hairpin structure will not block Dicer activity on the DsiRNA and will not interfere with the directed destruction of the target RNA.

In some examples, the antisense strand of the dsNAs is longer than the sense strand of the dsNA, resulting in the formation of a single-stranded overhang in the antisense strand. In some examples, the single-stranded overhang is at the 3′ terminus of the antisense strand. In some examples, the single-stranded overhang is of 1 to 10 nucleotides, or 2 to 9 nucleotides, or 3 to 8 nucleotides, or 4 to 7 nucleotides, or 5 to 6 nucleotides in length. In some specific examples, the single-stranded overhang is of 1 to 3 nucleotides in length. In one specific example, the single-stranded overhang is of 2 nucleotides in length. In some examples, the presence of the single-stranded overhang, particularly the single-stranded overhang of 2 nucleotides in length, promotes Dicer recognition for the dsNA.

In some examples, the nucleic acid or the dsNA as disclosed herein contain one or more modified nucleotides and/or one or more phosphate backbone modification.

In some examples, a modified nucleotide is modified at the 2′ position, the 3′ position, the 5′ position or the 6′ position of the sugar portion of the nucleotide, i.e. the modified nucleotide contains a modified sugar moiety. Examples of modified nucleotide include but are not limited to, deoxy-nucleotide, 3′-terminal deoxy-thymine (dT) nucleotide, 2′-O-methyl modified nucleotide, 2′-fluoro modified nucleotide, 2′-deoxy-modified nucleotide, locked nucleotide, unlocked nucleotide, conformationally restricted nucleotide, constrained ethyl nucleotide, abasic nucleotide, 2′-amino-modified nucleotide, 2′-O-allyl-modified nucleotide, 2′-C-alkyl-modified nucleotide, 2′-hydroxly-modified nucleotide, 2′-methoxyethyl modified nucleotide, 2′-O-alkyl-modified nucleotide, morpholino nucleotide, phosphoramidate, non-natural base comprising nucleotide, tetrahydropyran modified nucleotide, 1,5-anhydrohexitol modified nucleotide, cyclohexenyl modified nucleotide, nucleotide comprising a phosphorothioate group, nucleotide comprising a methylphosphonate group, nucleotide comprising a 5′-phosphate, nucleotide comprising a 5′-phosphate mimic, glycol modified nucleotide (GNA), 2-O-(N-methylacetamide) modified nucleotide, and combinations thereof. In one specific example, the modified nucleotide is 2′-O-methyl modified nucleotide.

Examples of phosphate backbone modification include but are not limited to, phosphorothioate modification, chiral phosphorothioate modification, phosphorodithioate modification, phosphotriester modification, aminoalkylphosphotriester modification, methyl or other alkyl phosphonate modification, phosphinate modification, phosphoramidate modification including 3′-amino phosphoramidate and aminoalkylphosphoramidate, thionophosphoramidate modification, thionoalkylphosphonate modification, thionoalkylphosphotriester modification, boranophosphate modification, and combinations thereof. In one specific example, the phosphate backbone modification is phosphorothioate modification.

In some examples, the oligonucleotide strand in the nucleic acid as disclosed herein, or the nucleic acid as disclosed herein, or at least one strand of the dsNA as disclosed herein, is attached to at least one moiety. Examples of such moiety include but are not limited to, a polyethylene glycol (PEG, either 20 kDa or 40 kDa) moiety, a cholesterol moiety, a dialkyl lipid, a protein, an organic or inorganic nanomaterial, and an inert antibody. Dialkyl lipids are lipids that contain two saturated or unsaturated alkyl chains, wherein each of the alkyl chains may be unsubstituted, or substituted by one or more aromatic, cycloaliphatic, or heterocyclic group(s). In some examples, each of the two alkyl chains contains at least 6, 8, 10, 12, 14, 16, 18, 20, 22 or 24 carbon atoms. Organic nanomaterials are prepared from natural or synthetic organic molecules or the mixture thereof. Organic nanomaterials are typically soft, biocompatible, biodegradable, less toxic, non-immunogenic, and highly stable. Examples of organic nanomaterials include but are not limited to, nanomicelles, liposomes, polymeric nanoparticles, dendrimers, exosomes and carbon nanomaterials (such as graphene oxide sheets, nanotubes, spherical shape fullerene). Inorganic nanomaterials are typically prepared synthetically from inorganic materials which are hard, water-insoluble, and less biodegradable. Examples of inorganic nanomaterial include but are not limited to, gold and iron oxide nanoparticles, mesoporous silica, hydroxyapatite, layered double hydroxides, carbon nanotubes, graphene, nanodiamonds, quantum dots, and lanthanide upconversion particles. Examples of inert antibodies include but are not limited to inert antibodies that target specific organs or tumors, or target tumor-specific surface receptors or neoantigens. Specific examples of inert antibodies that target tumors include but are not limited to anti-NaPi2b antibody, anti-LeY antibody, and anti-HER2 antibody. In some examples, the at least one moiety is attached to the 5′ end of the oligonucleotide strand or the 5′ end of the nucleic acid comprising the oligonucleotide strand. In some other examples, the at least one moiety is attached to the 5′ end of the antisense strand of the dsNA. In some examples, the addition of the at least one moiety provides the nucleic acid or the dsNA with one or more of the following characteristics: increased stability, increased half-life, increased bioavailability, reduced renal clearance, reduced in vivo toxicity, increased cellular uptake, and increased affinity to a specific cell type or a specific molecule.

In some examples, the oligonucleotide strand in the nucleic acid as disclosed herein, or the nucleic acid as disclosed herein, or at least one strand of the dsNA as disclosed herein, is attached to at least one moiety comprising an aptamer. Examples of aptamers include nucleic acid aptamers and peptide aptamers. In some examples, the aptamer is an anti-transferrin receptor (i.e. anti-TfR) aptamer. In some specific examples, the aptamer is an anti-human transferrin receptor (i.e. anti-hTfR) aptamer, such as an anti-hTfR1 or an anti-hTfR2 aptamer. In one specific example, the aptamer is a nucleic acid aptamer.

In some specific examples, the aptamer comprises a sequence which differs by 1, 2, or 3 nucleotides, or no more than 1, 2, or 3 nucleotides, from 5′-UGCGUUCACGUUUAUUCACAUUUUUGAAUUGAGCAUGAGC-3′ (SEQ ID NO: 15). In one specific example, the aptamer comprises a sequence which differs by no more than 3 nucleotides from SEQ ID NO: 15. In one specific example, the aptamer comprises or consists of the sequence 5′-GGGAGACAAGAAUAAACGCUCAAUGCGUUCACGUUUAUUCACAUUUUUGAAU UGAGCAUGAGCUUCGACAGGAGGCUCACAACAGGC-3′ (SEQ ID NO: 20). In some examples, the aptamer comprising a sequence which differs by 1, 2, or 3 nucleotides, or no more than 1, 2, or 3 nucleotides, from SEQ ID NO: 15, or the aptamer comprising a sequence which differs by no more than 3 nucleotides from SEQ ID NO: 15, or the aptamer comprising or consisting of SEQ ID NO: 20, is an anti-hTfR aptamer, more specifically an anti-hTfR1 or an anti-hTfR2 aptamer.

In some examples, the oligonucleotide strand in the nucleic acid as disclosed herein, or the nucleic acid as disclosed herein, or at least one strand of the dsNA as disclosed herein, is attached to an aptamer and at least one other moiety as described herein, such as a polyethylene glycol (PEG, either 20 kDa or 40 kDa) moiety, a cholesterol moiety, a dialkyl lipid, a protein, an organic or inorganic nanomaterial, and an inert antibody. In some examples, the at least one further moiety is attached to the 5′ end of the aptamer. In some other examples, the at least one further moiety is attached to the 3′ end of the aptamer.

In some examples, the oligonucleotide strand in the nucleic acid as disclosed herein, or the nucleic acid as disclosed herein, or at least one strand of the dsNA as disclosed herein, is attached to at least one moiety or the at least one further moiety as mentioned above via a linker. Suitable linker sequences would be readily apparent to one skilled in the art. For example, the linker can be a nucleotide linker, a non-nucleotide (i.e. chemical) linker, or combinations thereof. In some examples, the linker is a polynucleotide linker. A specific example of a polynucleotide linker is 5′-GUACAUUCUAGAUACGC-3′ (SEQ ID NO: 16). A non-nucleotide linker may be comprised of polyether, polyamine, polyamide, peptide, carbohydrate, lipid, polyhydrocarbon, or other polymeric compounds. In some examples, the linker is a non-nucleotide linker such as a hydrocarbon linker. The hydrocarbon linker can comprise a saturated cyclic group, a substituted saturated cyclic group, an aromatic cyclic group, a substituted aromatic cyclic group, or combinations thereof. In some examples, a hydrocarbon linker contains at least 2, 4, 6, 8, 10, 12, 14, 16, 18, 20, 22 or 24 carbon atoms. In one specific example, the hydrocarbon linker is a C3 carbon linker, i.e. a hydrocarbon linker with 3 carbon atoms, such as propane, propene or propyne linkers. In some examples, the hydrocarbon linker contains at least 1, 2, 3, 4, 5, 6, 7, 8, 9 or 10 C3 carbon linkers. In some specific examples, the hydrocarbon linker contains 5 or 7 C3 carbon linkers. In some examples, wherein the linker comprises a hydrocarbon linker and a polynucleotide linker, resulting in a nucleic acid or dsNA molecule comprising the structure of: [moiety]-[hydrocarbon linker]-[polynucleotide linker]-[oligonucleotide strand/antisense strand of the nucleic acid/dsNA] (each “[-]” represents a covalent bond. In some specific examples wherein the at least one moiety is an aptamer, the nucleic acid or dsNA molecule as disclosed herein comprises the structure of: [Aptamer]-[hydrocarbon linker]-[polynucleotide linker]-[oligonucleotide strand/antisense strand of the nucleic acid/dsNA].

In some examples, the nucleic acid or the dsNA as disclosed herein is formulated with poloxamer gel or a molecule to increase the molecular mass of the nucleic acid or the dsNA, to more than about 30, 35, 40, 45, 50, 55 or 60 kDa, or to more than about 30-50 kDa. In some examples, the molecular mass of about 30-50 kDa is the threshold for renal glomerulus clearance.

An agent comprising the nucleic acid or the dsNA as disclosed herein can be used for inhibiting expression of STAT3. Thus, provided herein also includes an agent comprising the nucleic acid or the dsNA as disclosed herein. Specifically, in one aspect, there is provided an agent for inhibiting expression of STAT3, comprising an aptamer and a double strand nucleic acid (dsNA), said dsNA comprising a sense strand and an antisense strand; wherein the aptamer is attached to one end of the antisense strand via a linker; wherein the aptamer comprises a sequence which differs by no more than 3 nucleotides from SEQ ID NO: 15; and wherein the antisense strand comprises at least 15 contiguous nucleotides differing by no more than 3 nucleotides from a nucleotide sequence selected from the group consisting of SEQ ID NO: 10 SEQ ID NOs: 12, and SEQ ID NOs: 14.

The nucleic acid, the dsNA, and/or the agent as disclosed herein can be further conjugated to or otherwise associated with a delivery vehicle such as a nanoparticle. Examples of nanoparticles include but are not limited to, lipid nanoparticles, polymer nanoparticles, inorganic nanoparticles, exosomes, and nucleic acid nanostructures. In one specific example, the nanoparticle is a lipid nanoparticle. In one example, a conjugated lipid nanoparticle includes the nucleic acid, the dsNA, and/or the agent as disclosed herein, a cationic lipid, a non-cationic lipid, and an aggregation reducing agent (such as PEG or PEG-modified lipid). In some examples, the nanoparticle is an exosome. In some specific examples, the exosome is a red blood cell derived extracellular vesicle (RBCEV), in particular a red blood cell derived exosome. Red blood cells (RBCs) release extracellular vesicles (EVs) including both endosome-derived exosomes and plasma-membrane-derived microvesicles (MVs). RBCEVs are secreted during erythropoiesis, physiological cellular aging, disease conditions, and in response to environmental stressors. RBCEVs are enriched in various bioactive molecules that facilitate cell to cell communication and can act as markers of disease. RBCEVs contribute towards physiological adaptive responses to hypoxia as well as pathophysiological progression of diabetes and genetic non-malignant hematologic disease.

In some examples, the nucleic acid, dsNA and/or agent as disclosed herein can be labeled by an appropriate moiety known in the art, for example, but not limited to one or more fluorophores, radioactive groups, chemical substituents, enzymes, antibodies or the like, to facilitate identification in hybridization assays and other assays or tests.

The nucleic acid, dsNA and/or agent provided herein can also be utilized in pharmaceutical compositions, by for example, adding an effective amount of the nucleic acid, dsNA and/or agent to a suitable pharmaceutically acceptable diluent or carrier.

Acceptable carriers and diluents are well known to those skilled in the art. Selection of a diluent or carrier is based on a number of factors, including, but not limited to, the solubility of the nucleic acid, dsNA and/or agent, and the route of administration. Such considerations are well understood by those skilled in the art.

The nucleic acid, dsNA and/or agent provided herein comprise any pharmaceutically acceptable salts, esters, or salts of such esters, or any other functional chemical equivalent which, upon administration to an animal including a human, is capable of providing (directly or indirectly) the biologically active metabolite or residue thereof. Accordingly, for example, the disclosure also provides prodrugs such as carboxylicacidesters prodrugs, monophosphate prodrugs, and amide-type prodrugs, and pharmaceutically acceptable salts of the nucleic acid, dsNA and/or agent, pharmaceutically acceptable salts of such prodrugs, and other bioequivalents.

The nucleic acid, dsNA and/or agent disclosed herein may also be admixed, encapsulated, conjugated or otherwise associated with other molecules, molecule structures or mixtures of compounds.

The pharmaceutical compositions may be administered in a number of ways depending upon whether local or systemic treatment is desired and upon the area to be treated.

The pharmaceutical formulations described herein, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers, finely divided solid carriers, or both, and then, if necessary, shaping the product (e.g., into a specific particle size for delivery).

A “pharmaceutical carrier” can be a pharmaceutically acceptable solvent, suspending agent or any other pharmacologically inert vehicle for delivering one or more nucleic acids to an animal and are known in the art. The carriers can be liquid or solid and is selected, with the planned manner of administration in mind, so as to provide for the desired bulk, consistency, etc., when combined with a nucleic acid and the other components of a given pharmaceutical composition. Liquid carriers can be aqueous carriers, non-aqueous carriers or both, and include, but are not limited to, aqueous suspensions, oil emulsions, water-in-oil emulsions, water-in-oil-in-water emulsions, site-specific emulsions, long-residence emulsions, sticky-emulsions, micro-emulsions and nano-emulsions. Solid carriers can be biological carriers, chemical carriers or both, and include, but are not limited to, viral vector systems, particles, microparticles, nanoparticles, microspheres, nanospheres, minipumps, bacterial cell wall extracts and biodegradable or non-biodegradable natural or synthetic polymers that allow for sustained release of the pharmaceutical compositions.

Preferred aqueous carriers include, but are not limited to, water, saline and pharmaceutically acceptable buffers. Preferred non-aqueous carriers include, but are not limited to, a mineral oil or a neutral oil including, but not limited to, a diglyceride, a triglyceride, a phospholipid, a lipid, an oil and mixtures thereof, wherein the oil contains an appropriate mix of polyunsaturated and saturated fatty acids. Examples include, but are not limited to, squalene, soybean oil, canola oil, palm oil, olive oil and myglyol, wherein the fatty acids can be saturated or unsaturated. Optionally, excipients may be included regardless of the pharmaceutically acceptable carrier. These excipients include, but are not limited to, anti-oxidants, buffers, and bacteriostats, and may include suspending agents and thickening agents.

The composition, shape, and type of dosage forms of the pharmaceutical composition as disclosed herein will typically vary depending on the intended use. For example, a dosage form used in the acute treatment of a disease or a related disease may contain larger amounts of one or more of the active compound it comprises than a dosage form used in the chronic treatment of the same disease. Similarly, a parenteral dosage form may contain smaller amounts of one or more of the active compound it comprises than an oral dosage form used to treat the same disease or disorder. These and other ways in which specific dosage forms encompassed by this invention will vary from one another will be readily apparent to those skilled in the art. Examples of dosage forms include, but are not limited to: tablets; caplets; capsules, such as soft elastic gelatine capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms particularly suitable for parenteral administration to a patient; and sterile solids (e.g., crystalline or amorphous solids) that can be reconstituted to provide liquid dosage forms suitable for parenteral administration to a patient. Thus, in one example, the pharmaceutical composition as disclosed herein is provided in a form selected from, but not limited to, tablets, caplets, capsules, hard capsules, soft capsules, soft elastic gelatine capsules, hard gelatine capsules, cachets, troches, lozenges, dispersions, suppositories, ointments, cataplasms, poultices, pastes, powders, dressings, creams, plasters, solutions, injectable solutions, patches, aerosols, nasal sprays, inhalers, gels, suspensions, aqueous liquid suspensions, non-aqueous liquid suspensions, oil-in-water emulsions, a water-in-oil liquid emulsions, solutions, sterile solids, crystalline solids, amorphous solids, solids for reconstitution or combinations thereof.

In one aspect, there is provided a method of treating cancer in a subject, comprising administering an effective amount of the nucleic acid, the dsNA, the agent, the nanoparticle, and/or the pharmaceutical composition as disclosed herein. In another aspect, there is provided the nucleic acid, the dsNA, the agent, the nanoparticle, and/or the pharmaceutical composition as disclosed herein for use in therapy. In another aspect, there is provided use of the nucleic acid, the dsNA, the agent, the nanoparticle, and/or the pharmaceutical composition as disclosed herein in the manufacture of a medicament for treating cancer.

Methods whereby bodily fluids, organs or tissues are contacted with an effective amount of one or more of the nucleic acid, the dsNA, the agent, the nanoparticle, and/or the pharmaceutical composition provided herein are also contemplated. Bodily fluids, organs or tissues can be contacted with one or more of the nucleic acid, the dsNA, the agent, the nanoparticle, and/or the pharmaceutical composition, resulting in modulation of the expression of STAT3 in the cells of bodily fluids, organs or tissues. An effective amount can be determined by monitoring the modulatory effect of the nucleic acid, the dsNA, the agent, the nanoparticle, and/or the pharmaceutical composition on STAT3 expression by methods routine to a person skilled in the art.

In some examples, the cancers to be treated by the nucleic acid, the dsNA, the agent, the nanoparticle, and/or the pharmaceutical composition as disclosed herein are cancers associated with STAT3 expression, STAT3 over-expression, and/or constitutive activation of STAT3 signaling. Examples of such cancers include but are not limited to, lung cancer, head and neck squamous cell carcinoma, endemic nasopharyngeal carcinoma, melanoma, breast carcinoma, prostate cancer, renal cell carcinoma, pancreatic carcinoma, ovarian cancer, leukemia, lymphoma and hepatocellular carcinoma (HCC). In one specific example, the lung cancer is a non-small cell lung cancer. Specific examples of leukemia include but are not limited to T-cell leukemia, large granular lymphocytic (LGL) leukemia, an acute myeloid leukemia (AML), and chronic myeloid leukemia (CML). Specific examples of lymphoma include but are not limited to anaplastic large cell lymphoma, and Burkitt's lymphoma.

In some examples, the methods of treating cancer as described herein comprise administration of plural therapeutic agents. In some examples, any nucleic acid, the dsNA, the agent, the nanoparticle, and/or the pharmaceutical composition as described herein is a first therapeutic agent, and the methods further comprises administering a second therapeutic agent. The second therapeutic agent can be administered before, concurrent or subsequent to the first therapeutic agent. In some examples, the second therapeutic is an RNA-based therapeutic or a small molecule drug.

It will be understood that a small molecule drug may refer to a drug known in the art for targeting cancer. Examples of appropriate drugs include: sorafenib, gefitinib, osimertinib, crizotinib, pemetrexed (Alimta), paclitaxel, carboplatin, gemcitabine, capecitabine, eribulin, 5-FU (5-fluorouracil) and others. Some drugs may be used in combination with oligonucleotides described herein to treat specific diseases or conditions. For example, when treating non-small cell lung cancer (NSCLC), the nucleic acid, the dsNA, the agent, the nanoparticle, and/or the pharmaceutical composition described herein may be combined with gefitinib, osimertinib (for EGFR mutants), crizotinib (for ALK mutants) or combinations thereof. In some examples, the nucleic acid, the dsNA, the agent, the nanoparticle, and/or the pharmaceutical composition as disclosed herein can be combined with a chemo drug such as pemetrexed (Alimta), which is used when tumors do not respond to targeted drugs. In cases where breast cancer is targeted, the nucleic acid, the dsNA, the agent, the nanoparticle, and/or the pharmaceutical composition as described herein may be combined with paclitaxel, carboplatin, gemcitabine, capecitabine, eribulin or combinations thereof. In cases where colon cancer is targeted, the nucleic acid, the dsNA, the agent, the nanoparticle, and/or the pharmaceutical composition as described herein may be combined with 5-FU (5-Flurouracil) or Capecitabine.

In some examples, in order to determine if the cancer in a patient is associated with STAT expression and thus should be treated with the nucleic acid, the dsNA, the agent, the nanoparticle, and/or the pharmaceutical composition as disclosed herein, a sample is obtained from the patient, in order to measure the level of STAT3 expression. Thus, in some examples, the method of treatment as disclosed herein further comprises measuring the level of STAT3 expression in a sample obtained from the subject, prior to administering a therapeutically effective amount of the nucleic acid, the dsNA, the agent, the nanoparticle, and/or the pharmaceutical composition as disclosed herein. In some examples, measuring the level of STAT3 expression comprises isolating and sequencing of RNA transcripts of STAT3.

In some examples, in order for the cancer in a patient to be considered as being associated with STAT3 expression, the level of STAT3 mRNA or Stat3 protein in the sample obtained from the patient is at least 15%, 20%, 25%, 30%, 35%, 40%, 45% or 50% higher as compared to the level of STAT3 mRNA or Stat3 protein in a sample obtained from a healthy subject.

In some examples, level of STAT3 expression is also determined during treatment to indicate efficacy of the treatment, and/or checked after treatment to determine if the treatment was effective.

The term “sample” used herein refers to a biological sample, or a sample that comprises at least some biological materials such as cells, DNAs or RNAs. Examples of biological samples include but are not limited to, solid tissue samples, such as bone marrow, and liquid samples, such as whole blood, blood serum, blood plasma, cerebrospinal fluid, central spinal fluid, lymph fluid, cystic fluid, sputum, stool, pleural effusion mucus, pleural fluid, ascitic fluid, amniotic fluid, peritoneal fluid, saliva, bronchial washes and urine. In some examples, the biological sample is a blood sample. In some other examples, the biological sample is a tumor sample obtained from tumor biopsies or surgically removed tumors.

The biological samples of this disclosure may be obtained from any organism, including mammals such as humans, primates (e.g., monkeys, chimpanzees, orangutans, and gorillas), cats, dogs, rabbits, farm animals (e.g. , cows, horses, goats, sheep, pigs), and rodents (e.g., mice, rats, hamsters, and guinea pigs).

It should be understood that following any method as described herein related to testing, identifying or screening, such methods may further comprise additional testing or screening for one or more additional genetic mutations, blood tests, blood enzyme tests, counseling, providing support resources or administering an additional pharmaceutical agent based on the results of such tests and/or screens. Similarly, it is further contemplated that such methods may be preceded by one or more steps, for example but not limited to selecting a subject that has cancer or thought to be at risk of having cancer, or selecting a subject that is pre-cancerous or suspected of being at risk for being pre-cancerous.

Methods of administration of the nucleic acid, the dsNA, the agent, the nanoparticle, and/or the pharmaceutical composition as disclosed herein include, but are not limited to the following: oral (e.g. buccal or sublingual), anal, rectal, as a suppository, intracolonic, topical, parenteral, nasal, aerosol, inhalation, intrathecal, intraperitoneal, intravenous, intra-arterial, transdermal, intradermal, subdermal, subcutaneous, intramuscular, intralymphatic, intrauterine, intravesicular, vaginal, visceral, into a body cavity, surgical administration at the location of the inflamed tissue such as adipose tissue, into the lumen or parenchyma of an organ, into bone marrow and into any mucosal surface of the gastrointestinal, reproductive, urinary and genitourinary system. It is to be understood that the choice of route of administration will be selected by one of ordinary skill in the art of treatment such that inhibition of or reduction in gene expression levels is achieved.

It is noted that, as used herein, the terms “organism”, “individual”, “subject”, or “patient” are used as synonyms and interchangeably.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation, or metabolites thereof, in the body of the patient. The administering physician can easily determine optimum dosages, dosing methodologies and repetition rates. Optimum dosages may vary depending on the relative potency of the composition, and can generally be estimated based on arithmetic means, for example based on ECso values found to be effective in in vitro and in vivo animal models, or based on the examples described herein. In general, dosage of the pharmaceutical composition according to the present disclosure is from about 0.01 μg to 100 g/kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the composition is administered in maintenance doses, ranging from 0.01 μg to 100 g/kg of body weight, once or more daily, to once every 2 years.

As mentioned above, a person skilled in the art would be able to ascertain, based on, for example, disease severity, the required dosage amount and dosage regime required to attain the desired clinical effect. The following is used as an illustrative example of an intravenous injection, which may be amended as required for other modes of administration. In one example, the method as disclosed herein requires at least one injection to be administered to the patient. In one example, more than a single injection may be administered to a patient at any given time. In yet another example, the method as disclosed herein may require that a single injection be administered to the patient more than once within a specified treatment timeframe or regime. In yet another example, the method as disclosed herein may require that more than two or more injections be administered to the patient more than once within a specified treatment timeframe or regime. This means, at according to clinical requirements, the patient may be given an initial treatment in the form of an injection, whereby further treatment may follow at interval of, for example, 3 day, 7 days, weekly, 2 weeks, fortnightly, 1 month, monthly, quarterly, biannually, annually or longer, depending on the treatment designed for the subject. If required, the method disclosed herein may also be used as in combination therapy with other drugs or pharmaceutical compositions.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

It will be understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase.

It will be understood by those skilled in the art that a wide variety of methods and techniques known in the art may be used in carrying out certain embodiments of the present invention.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non- limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

The following examples illustrate methods by which aspects of the invention may be practiced or materials suitable for practice of certain embodiments of the invention may be prepared.

### Example 1. Designing antisense sequence for specific inhibition of STAT3

Screening of Commercially Available STAT3 siRNAs

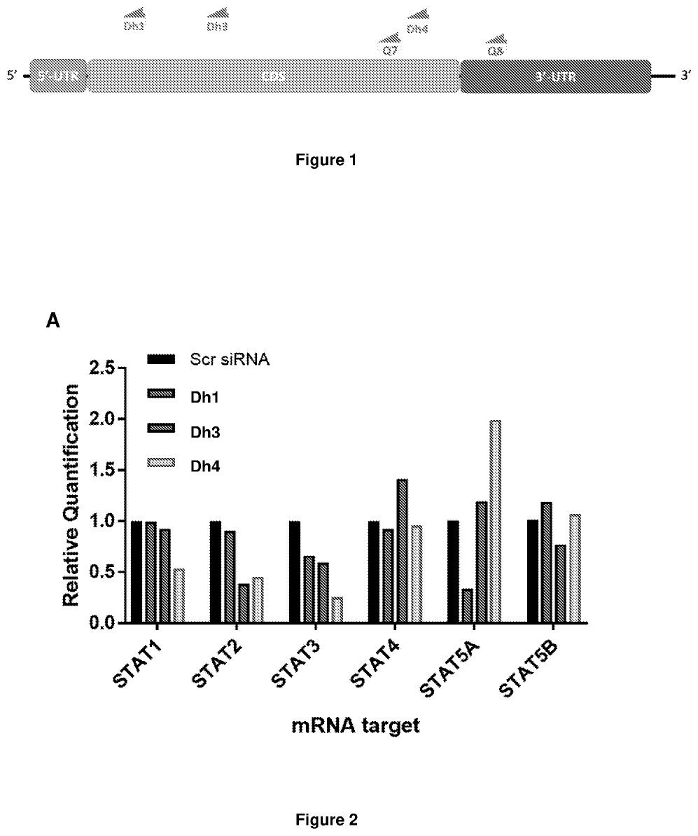

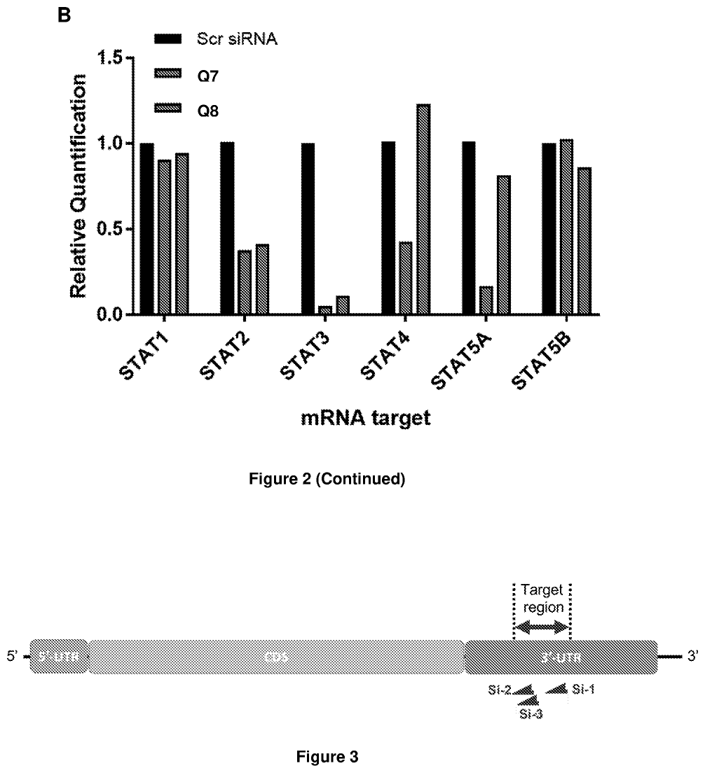

Commercially available STAT3 siRNA sequences (Dh1, Dh3, Dh4, Q7 and Q8) were obtained from two different sources (FIG. 1). The siRNA sequences are as follows: Dh1: 5′-GAGAUUGACCAGCAGUAUA-3′ (SEQ ID NO: 22), Dh3: 5′-CCAACAACCCAAGAAUGU-3′ (SEQ ID NO: 23), Dh4: 5′-15 CAACAGAUUGCCUGCAUUG-3′ (SEQ ID NO: 24), Q7: 5′-CAGCCTCTCTGCAGAATTCAA-3′ (SEQ ID NO: 25) and Q8: 5′-GGCUGGUAAUUUAUAUAAUTT-3′ (SEQ ID NO: 26). All sequences tested are proclaimed to be functionally validated in inhibiting STAT3. The siRNAs were transfected as per usual liposomal transfection protocol, and the mRNAs were harvested for quantification of STAT3 using RT-qPCR (FIG. 2). Despite obtaining decent knockdown of STAT3 mRNA (ranging from 33%-95% inhibition), each of these sequences has demonstrated non-specific inhibition of other STAT isoforms in H2170 cell line. Accordingly, a more robust and specific STAT3-targeting siRNA sequence needs to be designed.

DsiRNA targeting STAT3 3′-UTR is Highly Specific

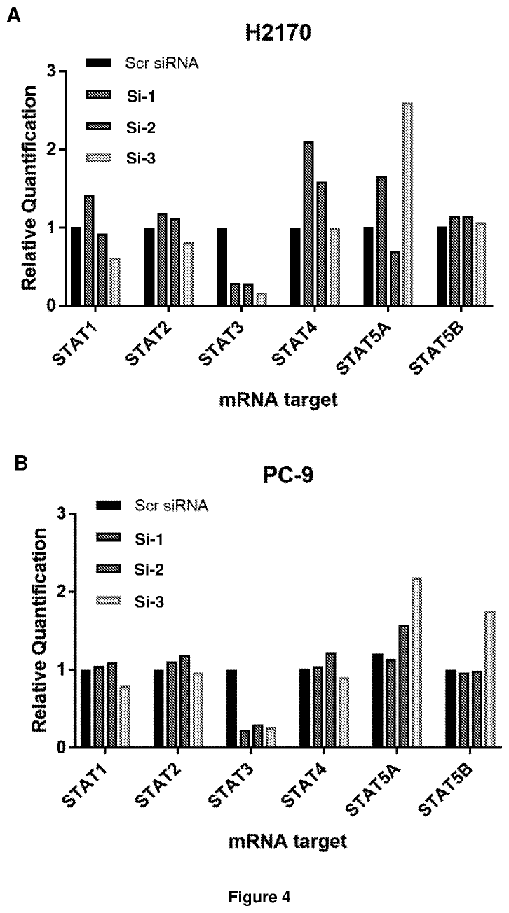

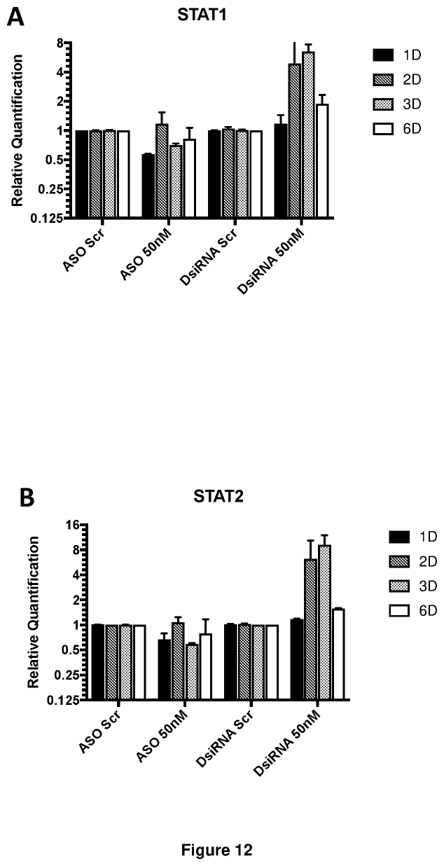

Despite the functional differences, the STAT proteins are considered to be highly homologous, with conserved domains that are critical for their functions. Therefore, targeting the coding region may not be effective. Accordingly, the inventors proposed to design siRNA directed at a region that is unique to STAT3 (FIG. 3). In order to increase the specificity, the inventors utilized a 27-mer DsiRNA instead of the usual 21-mer siRNA. Based on the preliminary data, and the general conception that 3′ UTR region is less conserved among genes within the same family, an antisense sequence targeting the 3′ UTR region of STAT3 was designed. FASTA sequence of the 3′ UTR of STAT3 (after the stop codon of the STAT3 gene) is as follows: 5′-GGAGCTGAGAACGGAAGCTGCAGAAAGATACGACTGAGGCGCCTACCTGCATTC TGCCACCCCTCACACAGCCAAACCCCAGATCATCTGAAACTACTAACTTTGTGGT TCCAGATTTTTTTTAATCTCCTACTTCTGCTATCTTTGAGCAATCTGGGCACTTTTA AAAATAGAGAAATGAGTGAATGTGGGTGATCTGCTTTTATCTAAATGCAAATAA GGATGTGTTCTCTGAGACCCATGATCAGG GGATGTG GC GGGGG GTGGCTAGAG G GAGAAAAAGGAAATGTCTTGTGTTGTTTTGTTCCCCTGCCCTCCTTTCTCAGCAGC TTTTTGTTATTGTTGTTGTTGTTCTTAGACAAGTGCCTCCTGGTGCCTGCGGCATC CTTCTGCCTGTTTCTGTAAGCAAATGCCACAGGCCACCTATAGCTACATACTCCT GGCATTGCACTTTTTAACCTTGCTGACATCCAAATAGAAGATAGGACTATCTAAG CCCTAGGTTTCTTTTTAAATTAAGAAATAATAACAATTAAAGGGCAAAAAACACT GTATCAGCATAGCCTTTCTGTATTTAAGAAACTTAAGCAGCCGGGCATGGTGGCT CACGCCTGTAATCCCAGCACTTTGGGAGGCCGAGGCGGATCATAAGGTCAGGAG ATCAAGACCATCCTGGCTAACACGGTGAAACCCCGTCTCTACTAAAAGTACAAA AAATTAGCTGGGTGTGGTGGTGGGCGCCTGTAGTCCCAGCTACTCGGGAGGCTG AGGCAGGAGAATCGCTTGAACCTGAGAGGCGGAGGTTGCAGTGAGCCAAAATTG CACCACTGCACACTGCACTCCATCCTGGGCGACAGTCTGAGACTCTGTCTCAAAA AAAAAAAAAAAAAAAAGAAACTTCAGTTAACAGCCTCCTTGGTGCTTTAAGCAT TCAGCTTCCTTCAGGCTGGTAATTTATATAATCCCTGAAACGGGCTTCAGGTCAA ACCCTTAAGACATCTGAAGCTGCAACCTGGCCTTTGGTGTTGAAATAGGAAGGTT TAAGGAGAATCTAAGCATTTTAGACTTTTTTTTATAAATAGACTTATTTTCCTTTG TAATGTATTGGCCTTTTAGTGAGTAAGGCTGGGCAGAGGGTGCTTACAACCTTGA CTCCCTTTCTCCCTGGACTTGATCTGCTGTTTCAGAGGCTAGGTTGTTTCTGTGGG TGCCTTATCAGGGCTGGGATACTTCTGATTCTGGCTTCCTTCCTGCCCCACCCTCC CGACCCCAGTCCCCCTGATCCTGCTAGAGGCATGTCTCCTTGCGTGTCTAAAGGT CCCTCATCCTGTTTGTTTTAGGAATCCTGGTCTCAGGACCTCATGGAAGAAGAGG GGGAGAGAGTTACAGGTTGGACATGATGCACACTATGGGGCCCCAGCGACGTGT CTGGTTGAGCTCAGGGAATATGGTTCTTAGCCAGTTTCTTGGTGATATCCAGTGG CACTTGTAATGGCGTCTTCATTCAGTTCATGCAGGGCAAAGGCTTACTGATAAAC TTGAGTCTGCCCTCGTATGAGGGTGTATACCTGGCCTCCCTCTGAGGCTGGTGAC TCCTCCCTGCTGGGGCCCCACAGGTGAGGCAGAACAGCTAGAGGGCCTCCCCGC CTGCCCGCCTTGGCTGGCTAGCTCGCCTCTCCTGTGCGTATGGGAACACCTAGCA CGTGCTGGATGGGCTGCCTCTGACTCAGAGGCATGGCCGGATTTGGCAACTCAA AACCACCTTGCCTCAGCTGATCAGAGTTTCTGTGGAATTCTGTTTGTTAAATCAA ATTAGCTGGTCTCTGAATTAAGGGGGAGACGACCTTCTCTAAGATGAACAGGGTT CGCCCCAGTCCTCCTGCCTGGAGACAGTTGATGTGTCATGCAGAGCTCTTACTTC TCCAGCAACACTCTTCAGTACATAATAAGCTTAACTGATAAACAGAATATTTAGA AAGGTGAGACTTGGGCTTACCATTGGGTTTAAATCATAGGGACCTAGGGCGAGG GTTCAGGGCTTCTCTGGAGCAGATATTGTCAAGTTCATGGCCTTAGGTAGCATGT ATCTGGTCTTAACTCTGATTGTAGCAAAAGTTCTGAGAGGAGCTGAGCCCTGTTG TGGCCCATTAAAGAACAGGGTCCTCAGGCCCTGCCCGCTTCCTGTCCACTGCCCC CTCCCCATCCCCAGCCCAGCCGAGGGAATCCCGTGGGTTGCTTACCTACCTATAA GGTGGTTTATAAGCTGCTGTCCTGGCCACTGCATTCAAATTCCAATGTGTACTTC ATAGTGTAAAAATTTATATTATTGTGAGGTTTTTTGTCTTTTTTTTTTTTTTTTTTTT TTGGTATATTGCTGTATCTACTTTAACTTCCAGAAATAAACGTTATATAGGAACC GTAAAAA-3′ (SEQ ID NO: 17). Using DsiRNA design tool provided by IDT, multiple potential DsiRNA were designed. Interestingly, three DsiRNA sequences were identified within a short 81nt target region (see FIG. 3) among the whole 3′ UTR region of STAT3, and this targeting region excludes the sequence of AZD9150 (5′-GCTGACATCCAAATAG-3′, SEQ ID NO: 18), an Antisense Oligo (ASO) marketed by AstraZeneca. Thus, this target region is a possible a hotspot for DsiRNA design. The target region was aligned to the homo sapiens genome using Blastn, and it was found that this target region is common to various STAT3 variants (FIG. 10). All three DsiRNA sequences (see Table 1) were synthesized for further validation.

TABLE 1
Sequences and properties of the scrambled DsiRNA, and STAT3-targeting
DsiRNAs.

| | | | Anhydrous | |
|---|---|---|---|---|
| | | | Molecular | |
| | | Sequence | Weight | GC Content |
| Scr- | Forward | rCrUrU rCrCrU rCrUrC rUrUrU | 7731.5 | |
| DsiRNA | | rCrUrC rUrCrC rCrUrU rGrUGA | | |
| | Reserve | rUrCrA rCrArA rGrGrG rArGrA | 8931.6 | |
| | | rGrArA rArGrA rGrArG rGrArA | | |
| | | rGrGrA | | |
| STAT3- | Forward | rArArG rGrUrU rUrArA rGrGrA | 8051 | 32 |
| DsiRNA1 | | rGrArA rUrCrU rArArG rCrAT T | | |
| | Reserve | rArArU rGrCrU rUrArG rArUrU | 8436.1 | 37.03704 |
| | | rCrUrC rCrUrU rArArA rCrCrU | | |
| | | rUrCrC | | |
| STAT3- | Forward | rArGrG rUrCrA rArArC rCrCrU | 7940.9 | 40 |
| DsiRNA2 | | rUrArA rGrArC rArUrC rUrGA A | | |
| | Reserve | rUrUrC rArGrA rUrGrU rCrUrU | 8612.1 | 40.74074 |
| | | rArArG rGrGrU rUrUrG rArCrC | | |
| | | rUrGrA | | |
| STAT3- | Forward | rGrGrU rCrArA rArCrC rCrUrU | 7956.9 | 44 |
| DsiRNA3 | | rArArG rArCrA rUrCrU rGrAA G | | |
| | Reserve | rCrUrU rCrArG rArUrG rUrCrU | 8588.1 | 44.44444444 |
| | | rUrArA rGrGrG rUrUrU rGrArC | | |
| | | rCrUrG | | |

STAT3 is often activated in cancer cells during oncogenesis. In this study, two cellular models have been selected: 1) A549 is a lung cancer cell line with high basal phosphorylated STAT3, and is commonly studied as a classical STAT3 model; and 2) PC-9 and its gefitinib-resistant counterpart, PC-9GR, which has acquired high STAT3 activity as an adaptive response to chronic gefitinib treatment. The efficacy of the customized DsiRNAs was determined by comparing the relative mRNA and protein expression of STAT3, comparing to other STAT isoforms, after transfection using standard protocol.

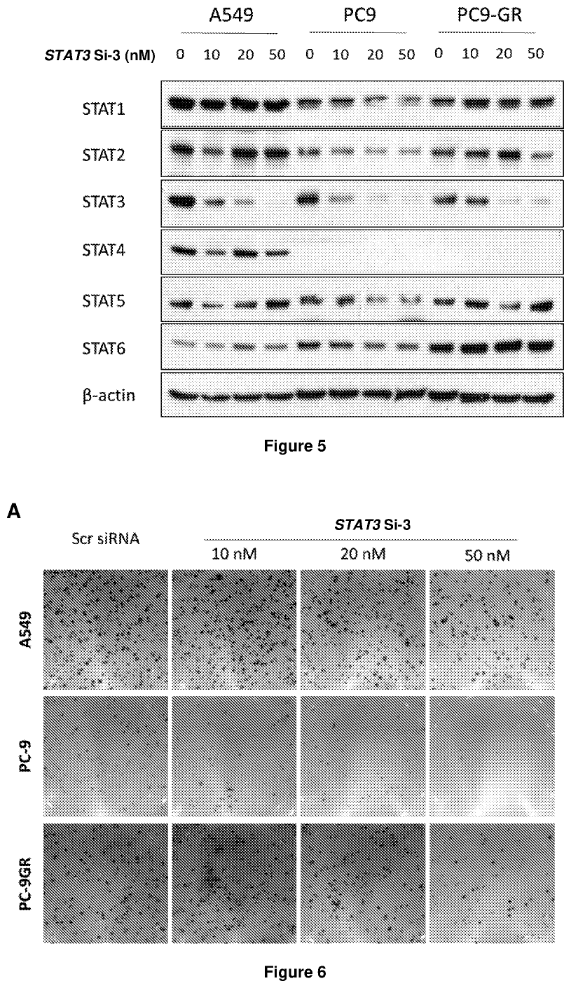

RT-qPCR was conducted post-transfection of control DsiRNA (i.e. Scr-DsiRNA) and the three customized STAT3-targeting sequences (STAT3-DsiRNA1, 2, 3, labelled as Si-1, Si-2 and Si-3 in the figures). In contrast to the commercial siRNAs, all three STAT3 DsiRNA sequences demonstrated enhanced specificity in the knockdown of STAT3 mRNA (FIG. 4). The specificity of the designed sequences was then compared to other pre-designed siRNAs. The knockdown efficiencies expressed as inhibition score (% inhibition of respective mRNA relative to Scrambled controls) of individual antisense sequences in H2170 were tabulated (Table 2). All three customized STAT3 DsiRNAs demonstrated potency to STAT3 (70.3% to 83.7%), with good knockdown rates that is comparable to Q8 (88.8%). More importantly, the customized STAT3 DsiRNAs exhibited strong selectivity to STAT3 inhibition (specificity score >50%), with minimal off target effects in other STAT isoforms (score <30%). On the contrary, all commercial siRNAs demonstrated off-target effects in other STAT isoform (specificity score >30%), and lack of specificity to STAT3 (Score <50%). STAT3 DsiRNA sequence 3 (STAT3-DsiRNA3) was selected for further validation.

TABLE 2
Tabulation of inhibition score (% reduction in the respective
mRNA relative to Scr controls) of STAT1, STAT2, STAT3, STAT4,
STAT5A, and STAT5B, to compare efficacies of antisense sequences
from commercial sources Q (Q7 and Q8) and Dh (Dh1, Dh3, and
Dh4), with customized STAT3 DsiRNA (1, 2, 3).

| | | | | | | | | |
|---|---|---|---|---|---|---|---|---|
| target | Q7 | Q8 | Dh1 | Dh3 | Dh4 | DsiRNA 1 | DsiRNA 2 | DsiRNA 3 |

Inhibition score

| | | | | | | | | |
|---|---|---|---|---|---|---|---|---|
| STAT1 | 9.5 | 5.4 | 0.8 | 7.5 | 46.3 | 0.0 | 7.7 | 38.5 |
| STAT2 | 62.4 | 58.7 | 9.4 | 61.3 | 54.7 | 0.0 | 0.0 | 18.6 |
| STAT3 | 94.9 | 88.8 | 33.7 | 40.5 | 74.8 | 70.3 | 71.4 | 83.7 |
| STAT4 | 57.2 | 0.0 | 7.7 | 0.0 | 4.4 | 0.0 | 0.0 | 0.7 |
| STAT5A | 83.2 | 18.5 | 66.3 | 0.0 | 0.0 | 0.0 | 30.4 | 0.0 |
| STAT5B | 0.0 | 13.9 | 0.0 | 22.8 | 0.0 | 0.0 | 0.0 | 0.0 |

Specificity score

| | | | | | | | | |
|---|---|---|---|---|---|---|---|---|
| Specificity | 3.1 | 2.9 | 0.6 | 5.7 | 25.7 | 0.0 | 7.0 | 27.2 |
| (STAT1) | | | | | | | | |
| Specificity | 20.3 | 31.7 | 8.0 | 46.4 | 30.3 | 0.0 | 0.0 | 13.2 |
| (STAT2) | | | | | | | | |
| Specificity | 30.9 | 47.9 | 28.6 | 30.7 | 41.5 | 100.0 | 65.2 | 59.2 |
| (STAT3) | | | | | | | | |
| Specificity | 18.6 | 0.0 | 6.5 | 0.0 | 2.4 | 0.0 | 0.0 | 0.5 |
| (STAT4) | | | | | | | | |
| Specificity | 27.1 | 10.0 | 56.3 | 0.0 | 0.0 | 0.0 | 27.8 | 0.0 |
| (STAT5A) | | | | | | | | |
| Specificity | 0.0 | 7.5 | 0.0 | 17.3 | 0.0 | 0.0 | 0.0 | 0.0 |
| (STAT5B) | | | | | | | | |

The shaded cells indicate significant decrease in mRNA.
Specificity score defined as (inhibition score of x)/(inhibition score of all STAT isoforms).
Numbers in bold indicate score > 50%; underlined numbers indicate score > 30%.

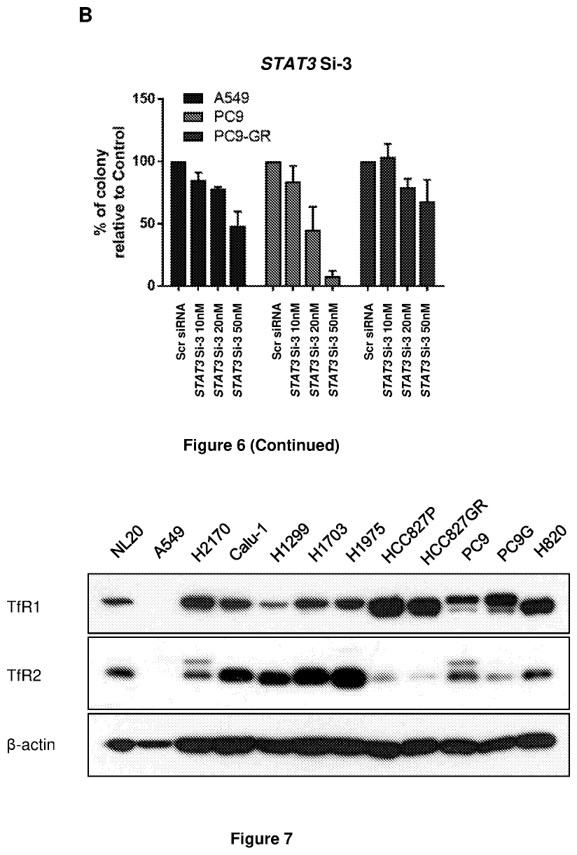

To validate that the silencing of STAT3 mRNA could affect the STAT3 protein expression in these cancer cell lines, the inventors next performed immunoblotting on A549, PC9 and PC9-GR cells transfected with increasing concentrations of STAT3-DsiRNA3. The efficacy and specificity of STAT3-DsiRNA3 was confirmed in all three cell lines, with strong inhibition of STAT33 protein but not of other STAT isoforms (FIG. 5).

STAT3 DsiRNA Inhibits Anchorage-Independent Growth

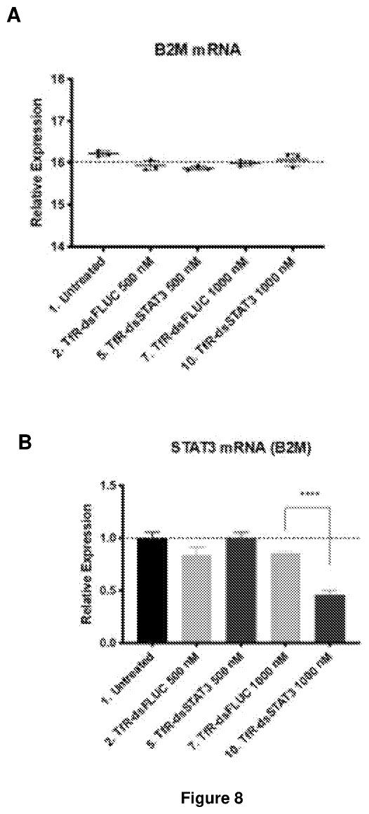

STAT3 signaling, when being constitutively active, can drive oncogenesis by promoting anchorage-independent growth—an attribute that favours malignant transformation and metastases. In this study, the inventors investigated the efficacy of the STAT3-DsiRNA3 in inhibiting colony formation in soft agar. As expected, cell lines with strong STAT3 activity (A549 and PC-9GR) formed large colonies (FIG. 6). As a proof-of-concept, there is a significant increase in colony counts in PC-9GR as compared to its parental counterpart, thus signifying the role of STAT3 in oncogenesis. Interestingly, the STAT3-DsiRNA3 was able to reduce colony formation in a dose-dependent manner, with the strongest effect observed at the highest dose across all cell types (FIG. 6).

### Example 2. Conjugating of STAT3-DsiRNA to Anti-hTfR Aptamer For Targeted Delivery

Aptamer and Transferrin Receptor (hTfR)

Target delivery using nanoparticles to increase therapeutic efficacy has been the focus of cancer therapeutics over the past decade. In this study, the inventors propose to increase target-specific homing of the STAT3-DsiRNAs using aptamer-conjugate. Aptamers are structured nucleic acid ligands with unique 3D structures based on defined nucleic acid sequences, selected in vitro using the systematic evolution of ligands by exponential enrichment (SELEX) strategy. Human transferrin receptor 1 (hTfR1) and 2 (hTfR2) are two receptors that were known to overexpressed. In addition, hTfR can be internalized into cancer cells through the clathrin-facilitated endocytosis pathway. Anti-hTfR aptamer has also been developed, characterized, and tested in principle with a small activation RNA targeting C/EBPα. Therefore, hTfR1 and hTfR2 were selected to develop an active targeting aptamer-conjugate.

Protein SELEX (Systemic Evolution of Ligands by Exponential Enrichment)

The extracellular domain of TfR was purchased from Sino Biological Inc (11020-HO7H). The SELEX in vitro selection was carried out as follows:

The 2′F-RNA aptamers were selected from randomized sequences. A random library of RNA oligonucleotides of sequence 5-GGGAGACAAGAATAAACGCTCAA-N40-TTCGACAGGAGGCTCACAACAGGC-3′ [N40 represents 40 nucleotide (nt) sequences formed by equimolar incorporation of A, G, C, and U at each position] (SEQ ID NO: 19) was constructed by in vitro transcription of synthetic DNA templates with NTPs (2′F UTP, 2′F CTP, GTP, ATP, Epicentre Biotechnologies, Madision, Wis.) and T7 RNA polymerase. To increase the nuclease resistance, 2′F-RNAs were used. To remove RNAs that bind nonspecifically to agarose beads, 1.44 μM of the RNA library was pre-incubated with 20 μl of Ni-NTA agarose beads in 100 μl binding buffer (30 mM Tris-HCl, 150 mM NaCl, 1.5 mM MgCl2, 2 mM dithiothreitol, and 1% BSA) for 30 mM at room temperature with shaking, precipitated by centrifugation, and discarded. The pre-cleared supernatant was transferred to a new tube and incubated with 333 nM of his-tagged TfR for 30 mM at room temperature. RNAs which bound to TfR were recovered, amplified by RT-PCR and in vitro transcription, and used in the following selection rounds. In subsequent rounds, capsid concentration was reduced by 2-fold at every 3 round for more stringent condition. After 12 rounds of SELEX, the resulting cDNA was amplified. The amplified DNA was cloned and individual clones were identified by DNA sequencing. Structures of aptamers were predicted using MFOLD (Zuker, M., Nucleic Acids Res., 31, 3406-3415 (2003)), available at http://www.bioinfo.rpi.edu/applications/mfold/, using a salt correction algorithm and temperature correction for 25° C.

A library of 2′F RNAs was used to increase nuclease-resistance and enhance aptamer folding. To isolate 2′F RNA aptamers binding to intact cells, a library of approximately 440 different 2′F RNA molecules, containing a 40-nt-long random sequence flanked by defined sequences, was screened by SELEX. After 12 cycles of selection, the highly enriched aptamer pools were cloned. The sequence of TR14 (5′-GGGAGACAAGAAUAAACGCUCAAUGCGUUCACGUUUAUUCACAUUUUUGAAU UGAGCAUGAGCUUCGACAGGAGGCUCACAACAGGC-3′, SEQ ID NO: 20) was selected to package with the STAT3 oligos.

hTfR1 and hTfR2 Are Expressed in Tumors and Cancer Cell Lines

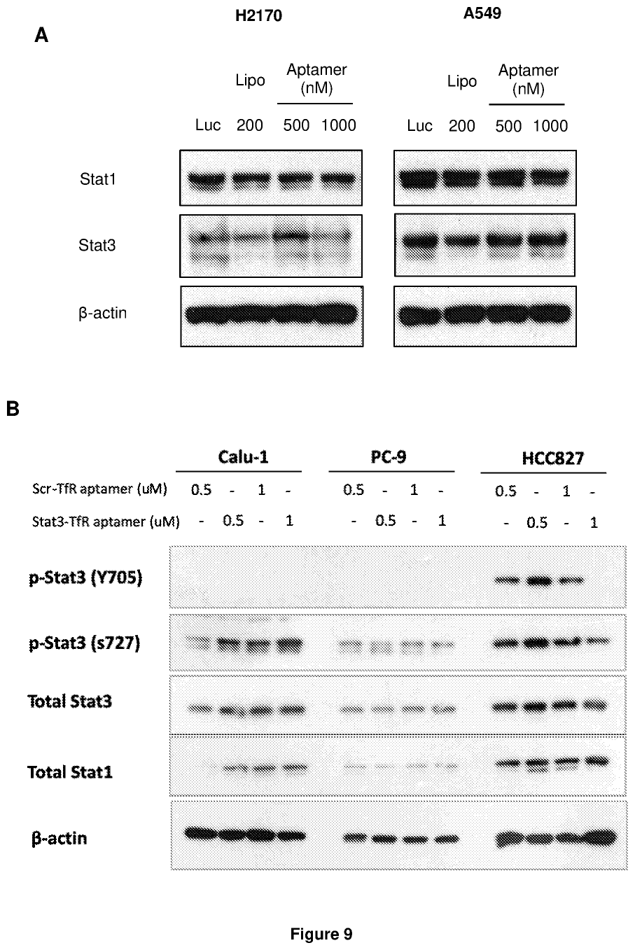

It was first determined whether TfR1 and TfR2 are expressed in a panel of patient-derived xenograft (PDX) tumors and cancer cell lines. Indeed, both hTfRs, particularly hTfR1, are found to be expressed in 50/109 of PDXs of hepatocellular tumor (data not shown), as well as 10/11 lung cancer cell lines (FIG. 7). Importantly, majority of the cancer cells have higher TfR1 than the normal lung epithelial cell line NL20 (FIG. 7).

Efficacy of STAT3-DsiRNA-hTfR Aptamer

The STAT3-DsiRNA3 was packaged into Anti-hTfR aptamer using the service of Apterna Co. (http://apterna.com). In order to maintain functional intergrity of the molecule, a “sticky sequence” was inserted between the TfR aptamer and either the STAT3-DsiRNA3 or control DsiRNA (antisense sequence targeting luciferase gene). The “sticky sequence” comprises a 16-nt sequence (GUACAUUCUAGAUACGC, SEQ ID NO: 16) that prevents structural hindrance of the molecule. It is inserted at the 3′ end of the TR14 aptamer following five C3 carbon linkers: 5′-NP40-UUCGACAGGAGGCUCACAACAGGC00000GUACAUUCUAGAUACGC-3′ (SEQ ID NO: 21).

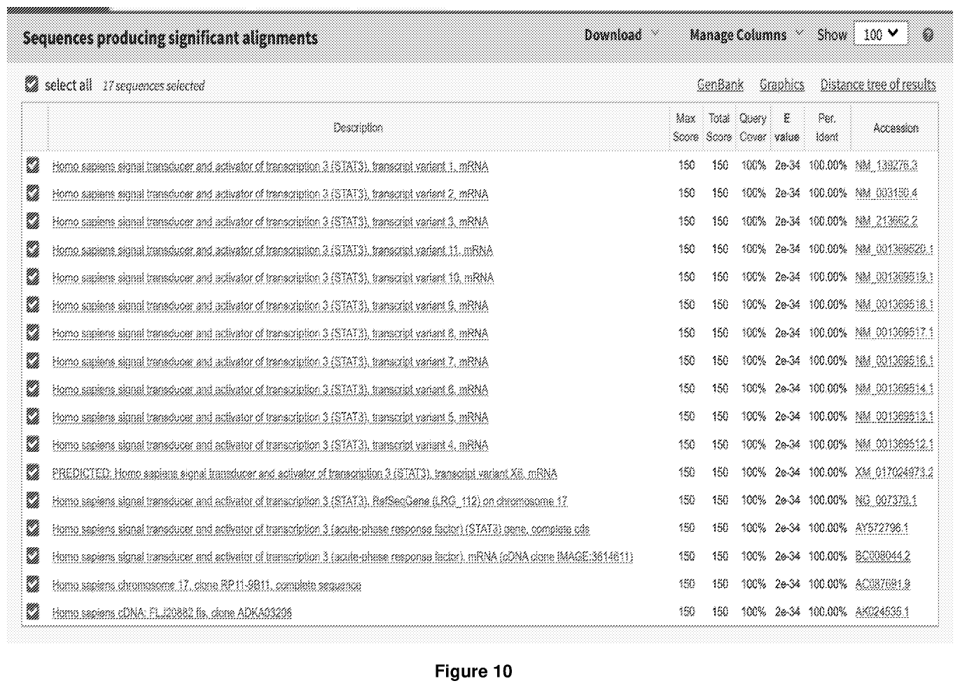

Active aptamers were packaged prior to each set of experiment in DPBS buffer using standard protocol. In brief, the aptamers were refolded in DPBS, heated to 94° C. for 5 min, slowly cooled to 37° C., and then incubated at 37° C. for 10 min The efficacy was first tested using a positive cell line, primary rat hepatocytes, that expressed hTfRs. RT-qPCR analysis revealed that both control and STAT3-DsiRNA-hTfR aptamers did not affect B2M gene (endogenous control) (FIG. 8A), with a 55% knockdown of STAT3 mRNA at 1 μM STAT3-hTfR aptamer (FIG. 8B).

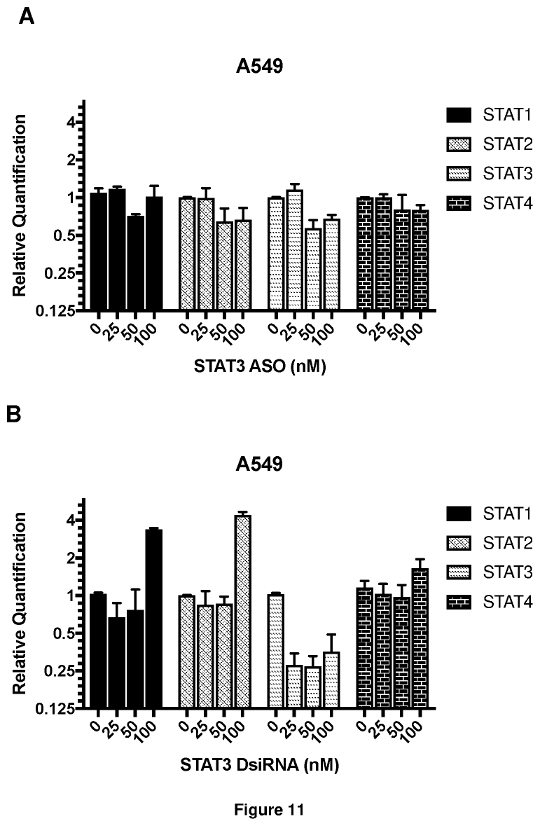

A two-step proof-of-concept approach was then conducted to validate the specificity of this delivery system. Firstly, a cancer cell line expressing TfRs (H2170) was transfected with both control and STAT3-DsiRNA-hTfR aptamer, and compared to a hTfR negative cell line (A549) (FIG. 7). Next, a human cancer cell line with moderate hTfR1 and hTfR2 (PC9), one with high hTfR1 (HCC827), and one expressing both hTfR1 and hTfR2 but not STAT3 Y705 phosphorylation (Calu-1) were selected. Each cell line was allowed to rest for 2 days to allow hTfR to be re-expressed after trypsinization. Control and STAT3-DsiRNA-hTfR aptamers were added twice with a 24 hours interval, and the cells were harvested for Western blotting. The result showed that STAT3-DsiRNA-hTfR aptamer had no effect on the hTfR-negative A549 cells, while reduction of Stat3 was observed in H2170 that is comparable to cells transfected with STAT3-DsiRNA-hTfR aptamer using JetPrime transfection protocol (FIG. 9A). It was also observed that STAT3-DsiRNA-hTfR aptamer strongly abrogated the phosphorylation of STAT3 at residue Y705, with marginal effect on residue 5727 (FIG. 9B).

In addition, a clear suppression of phosphorylated Stat3 was observed in HCC827 (exposure to 1 μM of STAT3-DsiRNA-hTfR aptamer significantly suppressed STAT3 tyrosine phosphorylation (Y705) in HCC827), but not so in PC9 cells (FIG. 9B), indicating that the aptamer has a higher selectivity towards hTfR1 than hTfR2. Taken together, these data indicated the specificity and selectivity of the STAT3-DsiRNA-hTfR aptamer in suppressing STAT3 activity in hTfR1-positive cancer cells.

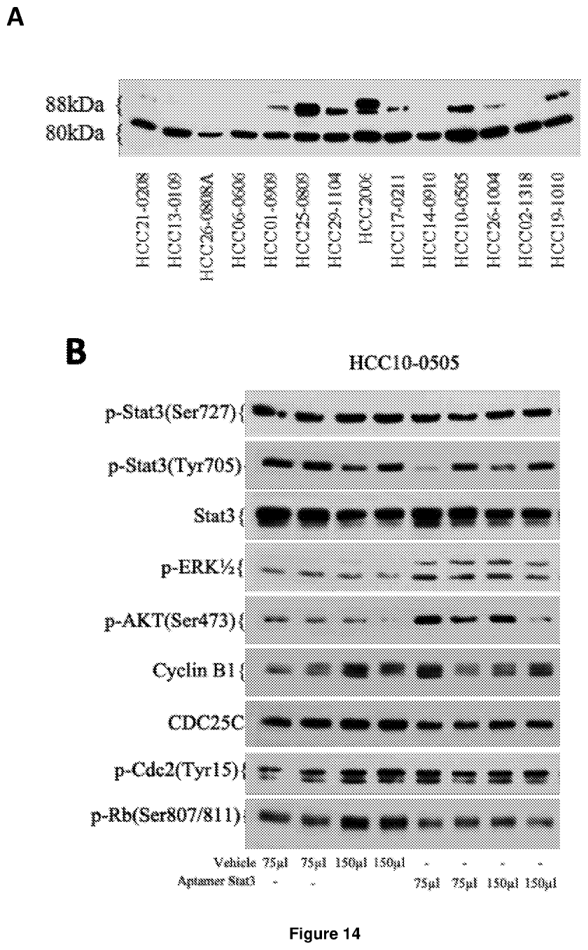

Activation of STAT3 is regulated by two main phosphorylated sites: (1) Residue Y705 that induces protein dimerization, nuclear translocation, and DNA binding; and (2) Residue S727 that triggers mitochondrial localization of STAT3 to regulate oxidative phosphorylation. The inventors showed that the selective inhibition of Y705-pSTAT3 by STAT3 aptamer did not interfere with mitochondrial respiration (OCR) (FIGS. 13A, B).

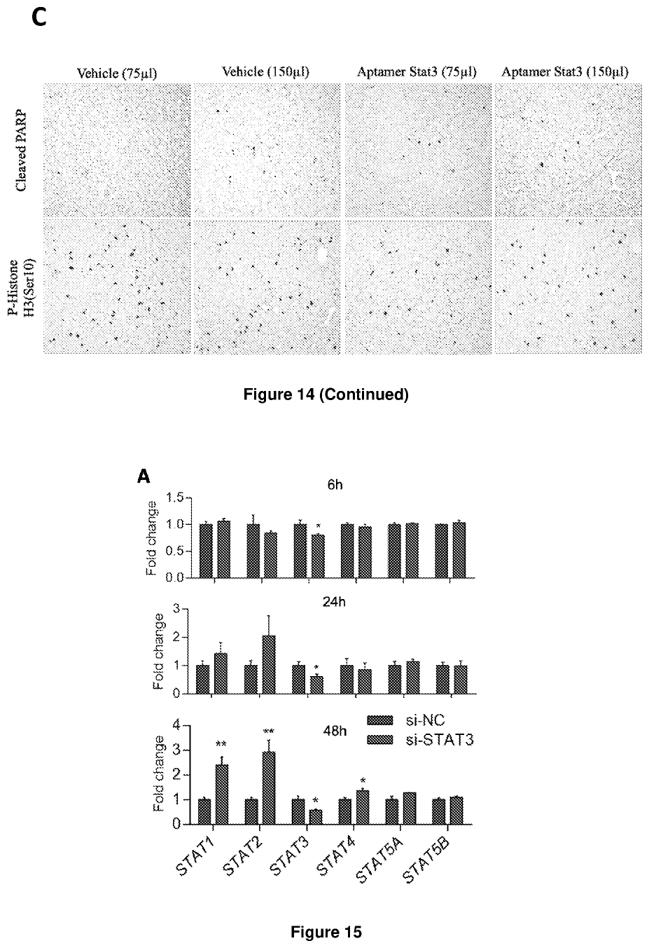

Aberrant STAT3 signaling has been reported as a main driver of hepatocellular carcinoma (HCC). As a proof-of-concept, the inventors proceeded to test the efficacy of STAT3-DsiRNA-hTfR aptamer in a patient-derived xenograft (PDX) of HCC tumour. More than 80 HCC PDX were screened and the population with high TfR1 expression was identified (representative image as FIG. 14A). A tumour with high TfR1 and strong STAT3 phosphorylation was selected for efficacy testing (HCC10-0505). In concordance with the in vitro observations (FIG. 7), STAT3- DsiRNA-hTfR aptamer strongly abrogated the phosphorylation of STAT3 at residue 705, with marginal effect on residue 727 (FIG. 14B). STAT3 inhibition did not exert apoptotic effects in the tumour cells (no apparent increase in cleaved PARP), but treatment with STAT3-DsiRNA-hTfR aptamer reduced cell proliferation as represented by Histone H3 Ser10 phosphorylation (FIG. 14C).

### Example 3. Enhanced Specificity, Potency and Stability of STAT3-DsiRNA

Comparison of the Specificity and Potency of STAT3 ASO and DsiRNA

Several STAT3-targeting RNA interference (RNAi) systems are currently being explored clinically. To verify the specificity, potency and stability of the STAT3-DsiRNAs as disclosed herein, the knockdown efficacy of the STAT3-DsiRNA3 was compared against AZD9150. As mentioned above, AZD9150 is a 16-mer single stranded ASO specific to STAT3. It is designed to target the 3′-UTR of STAT3 and has shown strong selectivity and specificity to this protein in several cancer models both in vitro and in vivo. The efficacy of AZD9150 has been explored clinically in diffuse large B cell lymphoma and hepatocellular carcinoma. While the STAT3-DsiRNAs as disclosed herein and AZD9150 are designed to target the 3′-UTR, the chemically synthesized 27-mer DsiRNAs as disclosed herein would have enhanced stability through the formation of RNA-induced silencing complex (RISC) with Dicer and AGO2. The carefully designed structure of the DsiRNAs was intended to improve Dicer processing and RISC loading. The dose- and time-dependent effects of STAT3-targeting ASO and DsiRNA were thus compared.

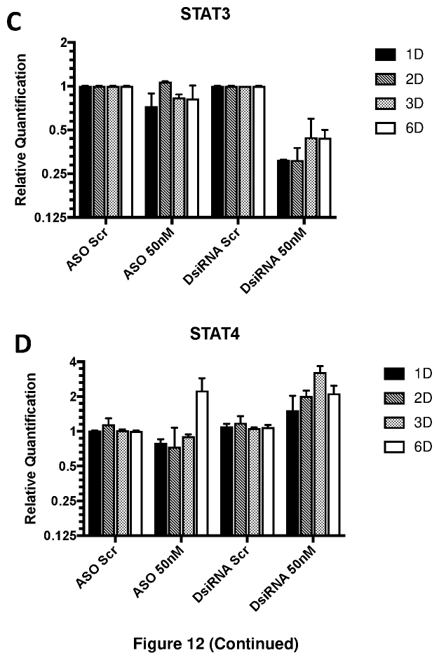

The results show that the STAT3-targeting ASO and DsiRNA3 have both demonstrated knockdown of STAT3 24 hours post transfection. However, AZD9150 (referred to as STAT3 ASO in FIG. 11A) was able to reduce STAT3 mRNA only at 50 nM (see FIG. 11A), as compared to 25 nM for STAT3-DsiRNA3 (see FIG. 11B). In addition, STAT3-DsiRNA3 could achieve a more robust knockdown (about 75% starting from 25 nM) as compared to STAT3-ASO (about 20%). Quantification of the nonspecific targeting of other STATs revealed a higher specificity of DsiRNA3 (59.9-100%) than ASO (33.8-37.2%) towards STAT3 (Table 3). 50nM of STAT3-ASO and STAT3-DsiRNA3 was selected for further investigation.

TABLE 3
Tabulation of inhibition score (% reduction in the
respective mRNA relative to Scr controls) of STAT1,
STAT2, STAT3, and STAT4 to compare efficacies of
STAT3-ASO (AZD9150) with customized STAT3-DsiRNA3
as disclosed herein at increasing concentrations
(25, 50 or 100 nM), 24 hours post-infection.

| | STAT3 ASO | | | STAT3 DsiRNA3 | | |
|---|---|---|---|---|---|---|
| target | 25 nM | 50 nM | 100 nM | 25 nM | 50 nM | 100 nM |
| STAT1 | 0.0 | 28.7 | 0.0 | 32.8 | 23.4 | 0.0 |
| STAT2 | 1.0 | 35.8 | 33.4 | 15.5 | 13.9 | 0.0 |
| STAT3 | 0.0 | 43.0 | 31.8 | 72.1 | 73.0 | 64.2 |
| STAT4 | 0.0 | 19.9 | 20.4 | 0.0 | 2.4 | 0.0 |

Specificity score

| | | | | | | |
|---|---|---|---|---|---|---|
| Specificity | 0.0 | 22.5 | 0.0 | 27.2 | 20.8 | 0.0 |
| (STAT1) | | | | | | |
| Specificity | 100.0 | 28.1 | 39.1 | 12.9 | 12.3 | 0.0 |
| (STAT2) | | | | | | |
| Specificity | 0.0 | 33.8 | 37.2 | 59.9 | 64.7 | 100.0 |
| (STAT3) | | | | | | |
| Specificity | 0.0 | 15.6 | 23.8 | 0.0 | 2.1 | 0.0 |
| (STAT4) | | | | | | |

The shaded cells indicate decrease in mRNA.
Specificity score defined as (inhibition score of x)/(inhibition score of all STAT isoforms).
Numbers in bold indicate score > 50%; underlined numbers indicates score > 30%.

Comparison of the Stability and Prolonged Potency of STAT3 ASO and DsiRNA

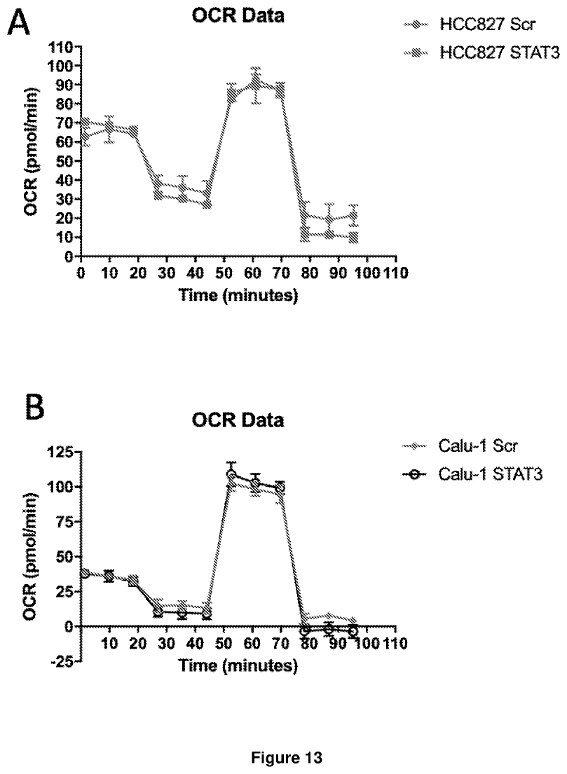

A549 cells were transfected with 50nM of the control, STAT3-ASO or STAT3-DsiRNA3, and cells were harvest at 4 different time points (Day 1, 2, 3, and 6). The mRNA level of STAT1, STAT2, STAT3 and STAT4 were measured and compared relative to the scrambled RNAi at the respective time point. Consistent with the observation in FIG. 11, STAT3-DsiRNA3 demonstrated a superior specificity and potency than STAT3-ASO starting from Day 1, as indicated by the drop in STAT3 mRNA (FIG. 12C). The strong knockdown efficacy could be sustained up to Day 6 (from about 70% at Day 1 to about 55% at Day 6), as compared to that of STAT3-ASO (from about 30% at Day 1 to about 20% at Day 6). This suggests that STAT3-DsiRNA3 is able to sustain the potent STAT3 inhibition, likely through the formation of RISC, whereas the single-stranded STAT3-ASO could be removed by intracellular nuclease.

Next, the non-specific effect of both STAT3-ASO and STAT3-DsiRNA3 on STAT1, STAT2 and STAT4 was investigated. Relative to Scr control, STAT3-DsiRNA3 demonstrated minimal off-target efficacy (see FIG. 12A, B, D and Table 4). Instead, STAT3-DsiRNA3 induced up-regulation of STAT1 and STAT2 at Day 2 and Day 3. This result was not surprising, as STAT1 and STAT3 are known to play opposite roles in tumorigenesis. While STAT3 is pro-survival, STAT1 triggers pro-apoptotic response and promotes innate/adaptive immunity. This provides additional benefit for the STAT3-DsiRNA. In contrast, STAT3-ASO showed non-specific knockdown of STAT1, 2 and 4 to various extent across the few time points tested (see FIGS. 12A, B, D).

TABLE 4
Tabulation of inhibition score (% reduction in the respective mRNA
relative to Scr controls) of STAT1, STAT2, STAT3, and STAT4 to
compare efficacies of AZD9150 (STAT3-ASO) and customized STAT3-
DsiRNA3 at 50 nM concentration, 1/2/3/6 Days post-infection.

| | STAT3 ASO | | | | STAT3 DsiRNA3 | | | |
|---|---|---|---|---|---|---|---|---|
| | 1 Day | 2 Days | 3 Days | 6 Days | 1 Day | 2 Days | 3 Days | 6 Days |
| STAT1 | 42.85 | 0 | 29.3 | 18.2 | 0 | 0 | 0 | 0 |
| STAT2 | 33.15 | 0 | 41.15 | 21.6 | 0 | 0 | 0 | 0 |
| STAT3 | 27.4 | 0 | 16.55 | 17.75 | 68.9 | 68.95 | 55.55 | 55.95 |
| STAT4 | 20.7 | 26.65 | 9.15 | 0 | 0 | 0 | 0 | 0 |

Specificity score

| | | | | | | | | |
|---|---|---|---|---|---|---|---|---|
| Specificity | 34.5 | 0.0 | 30.5 | 31.6 | 0.0 | 0.0 | 0.0 | 0.0 |
| (STAT1) | | | | | | | | |
| Specificity | 26.7 | 0.0 | 42.8 | 37.5 | 0.0 | 0.0 | 0.0 | 0.0 |
| (STAT2) | | | | | | | | |
| Specificity | 22.1 | 0.0 | 17.2 | 30.8 | 100.0 | 100.0 | 100.0 | 100.0 |
| (STAT3) | | | | | | | | |
| Specificity | 16.7 | 100.0 | 9.5 | 0.0 | 0.0 | 0.0 | 0.0 | 0.0 |
| (STAT4) | | | | | | | | |

The shaded cells indicate decrease in mRNA.
Specificity score defined as (inhibition score of x)/(inhibition score of all STAT isoforms).
Numbers in bold indicate score > 50%; underlined numbers indicate score > 30%.

### Example 4. Efficacy of STAT3 DsiRNA In Red Blood Cell Derived Exosomes (RBCEVs)

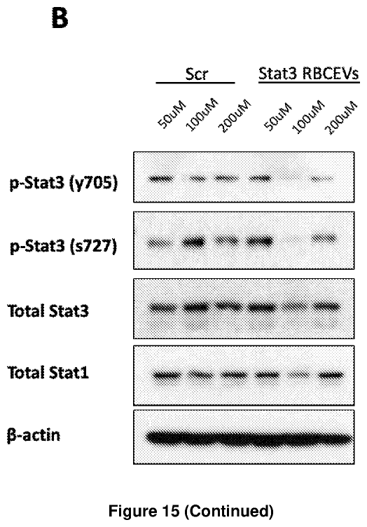

Given that transferrin receptor 1 expression is not expressed in all cancer cells, the inventors have explored the possibilities of packaging STAT3-DsiRNA sequence in other nanoparticles which are conjugate-free, in order to enhance delivery to cancer cells. First, the oligo sequences were packaged into red blood cell derived exosomes (RBCEVs), a delivery vessel that has shown promising efficacy with other antisense oligos. As shown in FIG. 15A, STAT3-DsiRNA-RBCEV was able to reduce STAT3 mRNA level (quantitative real-time PCR) as early as 6 h in lung cancer A549 cells. Maximum efficacy of ˜50% was achieved 24 h post treatment, with limited non-specific effects on other STAT isoforms. In addition, the effect of STAT3-DsiRNA-RBCEV was able to last for 48 h. The efficacy on reducing STAT3 protein was also validated using western blotting (FIG. 15B).

1.-38. (canceled)

39. A nucleic acid comprising an oligonucleotide strand of 15-80 nucleotides in length, wherein said oligonucleotide strand is at least 80% complementary to a target STAT3 mRNA sequence along at least 15 nucleotides of said oligonucleotide strand, wherein the target STAT3 mRNA sequence has the sequence of SEQ ID NO: 1.

40. A double stranded nucleic acid (dsNA) for inhibiting expression of STAT3, comprising a sense strand and an antisense strand forming a double stranded region, wherein the antisense strand is at least 80% complementary to a target STAT3 mRNA sequence along at least 15 nucleotides of said antisense strand, wherein the target STAT3 mRNA sequence has the sequence of SEQ ID NO: 1; and wherein the sense strand is at least 80% complementary to a sequence provided in SEQ ID NO: 2, along at least 15 nucleotides of said sense strand.

41. The dsNA of claim 40, wherein the sense strand comprises at least 15 contiguous nucleotides that are at least 80% complementary to a nucleotide sequence selected from the group consisting of SEQ ID NO: 4, SEQ ID NOs: 6, and SEQ ID NOs: 8; and wherein the antisense strand comprises at least 15 contiguous nucleotides that are at least 80% complementary to a nucleotide sequence selected from the group consisting of SEQ ID NO: 3, SEQ ID NOs: 5, and SEQ ID NOs: 7.

42. The dsNA of claim 40, wherein the sense strand comprises at least 15 contiguous nucleotides differing by no more than 3 nucleotides from a nucleotide sequence selected from the group consisting of SEQ ID NO: 9, SEQ ID NOs: 11, and SEQ ID NOs: 13; and wherein the antisense strand comprises at least 15 contiguous nucleotides differing by no more than 3 nucleotides from a nucleotide sequence selected from the group consisting of SEQ ID NO: 10 SEQ ID NOs: 12, and SEQ ID NOs: 14.

43. The dsNA of claim 40, wherein the sense strand is 15-80 nucleotides in length and the antisense strand is 19-80 nucleotides in length; or wherein the sense strand is nucleotides in length and the antisense strand is 19-35 nucleotides in length.

44. The dsNA of claim 40, wherein the dsNA comprises a duplex region selected from the group consisting of 19, 20, 21, 22, 23, 24, 25, 26, or 27 base pairs.

45. The dsNA of claim 40, wherein the antisense strand comprises 1-5 single-stranded nucleotides at its 3′ terminus, optionally 1-3 nucleotides in length, and optionally 2 nucleotides in length.

46. The dsNA of claim 40, comprising at least one modified nucleotide, wherein the at least one modified nucleotide is modified at the 2′ position, the 3′ position, the 5′ position or the 6′ position; optionally wherein the at least one modified nucleotide is selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide (GNA), and a 2-O-(N-methylacetamide) modified nucleotide; and combinations thereof.

47. The dsNA of claim 40, comprising a phosphate backbone modification selected from the group consisting of a phosphorothioate, a chiral phosphorothioate, a phosphorodithioate, a phosphotriester, an aminoalkylphosphotriester, a methyl or other alkyl phosphonate, a phosphinate, a phosphoramidate including 3′-amino phosphoramidate and aminoalkylphosphoramidate, a thionophosphoramidate, a thionoalkylphosphonate, a thionoalkylphosphotriester, and a boranophosphate.

48. The dsNA of claim 40, wherein the oligonucleotide strand, or at least one strand of the dsNA is attached to at least one moiety, optionally wherein the addition of the at least one moiety provides the nucleic acid or the dsNA with one or more of the following characteristics: increased stability/increased half-life, increased bioavailability, reduced renal clearance, reduced in vivo toxicity, increased cellular uptake, and increased affinity to a specific cell type or a specific molecule; optionally wherein the at least one moiety is selected from the group consisting of a polyethylene glycol (PEG), a cholesterol, a dialkyl lipid, a protein, an organic or inorganic nanomaterial, and an inert antibody, and wherein the at least one moiety is at the 5′ end of the oligonucleotide strand or the antisense strand.

49. The dsNA of claim 40, wherein the nucleic acid or the dsNA is formulated with poloxamer gel or a molecule to increase the molecular mass of the nucleic acid or the dsNA to more than about 30-50 kDa.

50. The dsNA of claim 48, wherein the at least one moiety comprises an aptamer; optionally wherein the aptamer is an anti-transferrin receptor (anti-TfR) aptamer, optionally an anti-human transferrin receptor (anti-hTfR) aptamer.

51. The dsNA of claim 50, wherein the aptamer comprises a sequence which differs by no more than 3 nucleotides from SEQ ID NO: 15.

52. The dsNA of claim 48, wherein the moiety is attached to the oligonucleotide strand or the antisense strand via a linker; optionally wherein the linker comprises a hydrocarbon linker, wherein the hydrocarbon linker is optionally a C3 carbon linker, or wherein the linker comprises a polynucleotide linker; optionally wherein the polynucleotide linker comprises the sequence of SEQ ID NO: 16.

53. The dsNA of claim 52, wherein the linker comprises a hydrocarbon linker and a polynucleotide linker, resulting in a molecule comprising the structure of: [moiety]-[hydrocarbon linker]-[polynucleotide linker]-[oligonucleotide strand/antisense strand of the nucleic acid/dsNA], wherein each “[-]” represents a covalent bond; optionally wherein the molecule comprises the structure of: [Aptamer]-[hydrocarbon linker]-[polynucleotide linker]-[oligonucleotide strand/antisense strand of the nucleic acid/dsNA].

54. An agent for inhibiting expression of STATS, comprising an aptamer and a double strand nucleic acid (dsNA), said dsNA comprising a sense strand and an antisense strand; wherein the aptamer is attached to one end of the antisense strand via a linker; wherein the aptamer comprises a sequence which differs by no more than 3 nucleotides from SEQ ID NO: 15; and wherein the antisense strand comprises at least contiguous nucleotides differing by no more than 3 nucleotides from a nucleotide sequence selected from the group consisting of SEQ ID NO: 10 SEQ ID NOs: 12, and SEQ ID NOs: 14.

55. A nanoparticle comprising the dsNA of claim 40.

56. The nanoparticle of claim 55, wherein the nanoparticle is a lipid nanoparticle, a polymer nanoparticle, an inorganic nanoparticle, an exosome, or a nucleic acid nanostructure.

57. A method of treating cancer in a subject, said method comprising administering an effective amount of the dsNA of claim 40.

58. The method of claim 57, wherein the cancer is selected from the group consisting of: a lung cancer, a head and neck squamous cell carcinoma, an endemic nasopharyngeal carcinoma, a melanoma, a breast carcinoma, a prostate cancer, a renal cell carcinoma, a pancreatic carcinoma, ovarian cancer, a leukemia, a lymphoma and a hepatocellular carcinoma (HCC).
